(19) United States
(12) Patent Application Publication
(10) Pub. No.: US 2025/0221932 A1
(43) Pub. Date: Jul. 10, 2025

(54) IMMUNE ENHANCEMENT OF CANCER TREATMENT

(71) Applicants: Spark Therapeutics, Inc., Philadelphia, PA (US); Institut d'Investigacions Biomédiques August Pi i Sunyer (IDIBAPS), Barcelona (ES)

(72) Inventors: Xavier ANGUELA, Barcelona (ES); Sean ARMOUR, Holland, PA (US); Pedro CEJAS, Oreland, PA (US); Sonia GUEDAN, Barcelona (ES); Ali NAHVI, Needham, MA (US); Seoyun YUM, Philadelphia, PA (US); Rui ZHANG, Winchester, MA (US)

(21) Appl. No.: 18/853,244

(22) PCT Filed: Mar. 31, 2023
(86) PCT No.: PCT/US2023/017109

§ 371 Date: Oct. 1, 2024

Related U.S. Application Data
(60) Provisional application No. 63/362,449, filed on Apr. 4, 2022.

(51) Int. Cl. A61K 9/1272; A61K 9/51; A61K 31/713; A61K 45/06; A61P 35/02

(52) U.S. Cl. CPC A61K 9/1272; A61K 9/5123; A61K 31/713; A61K 45/06; A61P 35/02

(57) ABSTRACT

The present invention features methods utilizing nanoparticles for double-stranded DNA (dsDNA). The nanoparticles are able to deliver the dsDNA intracellularly where the dsDNA can stimulate the innate immune response. The provided methods can be used to treat cancer and be utilized in combination with different types of therapeutic agents having anti-cancer activity and with cancer vaccines.

### FIELD OF THE INVENTION

The invention relates to methods for enhancing an immune response and methods of treating cancer. The provided methods can be used, for example, in combination with different types of therapeutic agents having anti-cancer activity and with cancer vaccines.

### BACKGROUND OF THE INVENTION

Cancer results from uncontrolled division of abnormal cells and can develop throughout the body. Once formed in a particular location, the cancer may be able to spread to other parts of the body. Two general types of cancer are blood cancers such as leukemia, lymphoma and multiple myeloma; and solid tumors that effect body organs or tissues. (See, e.g., hypertext transfer protocol://www.cancer.org/treatment/understanding-your-diagnosis/what-is-cancer.html.)

Tumors are generally characterized by mutated cells having uncontrolled proliferation that lump together with immune, endothelial and mesenchymal cells, and their extracellular matrix, forming a tumor immune microenvironment. Based on the microenvironment, the tumor can be characterized as hot or cold. Hot tumors, compared to cold tumor, are characterized by a (i) higher infiltration of T cells within the tumor and at the invasive margin and (ii) a higher level of proinflammatory cytokines. Hot tumors are more responsive to immune-checkpoint blockade than cold tumors. (Galon and Bruni (2019) Nature Reviews 18:197-217.)

Tumor types can be stratified utilizing an immune score of 10 (cold) to 14 (hot) based on quantification of various CD3+ lymphocyte populations. (Pagès et al., (2018) Lancet 391 2128-2139; and Galon and Bruni (2019) Nat. Rev. Drug Discov. 18:197-218.)

References discussing cancer treatment involving possible ways to convert cold to hot tumors include: Liu and Sun (2021) Theranostics; 11 (11): 5365-5386; and Liang et al., (2020), Sci. Adv. 6: eabc3646 28 Aug. 2020.)

### BRIEF SUMMARY OF THE INVENTION

The present invention features methods utilizing nanoparticles for delivering a double-stranded DNA (dsDNA). The nanoparticles are able to deliver the dsDNA intracellularly where the dsDNA can stimulate the innate immune response. Uses of the described methods include cancer treatment.

Thus, a first aspect of the present invention describes a method of treating a cancer in a subject, comprising administering to the subject: (a) a nanoparticle comprising a dsDNA; and (b) a cancer vaccine or a cancer therapeutic agent. In an embodiment, the dsDNA comprises a dsDNA region at least 45 base pairs in length.

A cancer vaccine provides an antigen to which an immune response is directed. A variety of different cancer vaccines can be used including those directly providing an antigen such as a tumor associated antigen or tumor specific antigen and nucleic acid encoding a polypeptide tumor associated antigen or tumor specific antigen.

The cancer therapeutic agent provides a compound having activity against the cancer and/or boosting the host immune response against cancer. Reference to compound or agent is not a limitation as to the size or complexity of the compound. Examples of compounds and agents include small molecules and larger molecules such as antibodies and other proteins.

Another aspect of the present invention describes a method of treating cancer in a subject, comprising administering to the subject a nanoparticle comprising a dsDNA. In different embodiments the cancer is lung cancer, melanoma, leukemia, or liver cancer; and/or the dsDNA comprises a dsDNA region at least 45 base pairs in length.

Another aspect, describes a method of treating a cancer in a subject comprising administering to the subject:
a) a nanoparticle comprising a double-stranded DNA (dsDNA), wherein the dsDNA comprises a double-stranded region of at least 45 base pairs in length; and
b) a checkpoint inhibitor;
   wherein (i) the cancer is resistant to checkpoint inhibitor treatment, (ii) the subject has previously undergone a treatment with a checkpoint inhibitor in the absence of a dsDNA-nanoparticle treatment, (iii) the subject either had no response to a prior checkpoint inhibitor treatment in the absence of a dsDNA-nanoparticle or a decreasing level of response to the prior treatment, and/or (iv) said cancer is stratified I3 or 14 based on quantification of CD3+ and CD8+ lymphocyte populations.

Another aspect is directed to a lipid nanoparticle comprising a double-stranded DNA (dsDNA), wherein said dsDNA comprises a double-stranded region of at least 45 base pairs in length and said dsDNA is non-coding or lacks a promoter operatively linked to a region coding for expression in the subject.

Additional aspects are directed to a nanoparticle comprising a dsDNA, for use in the methods described herein; and use of a nanoparticle comprising a dsDNA, for the preparation of a medicament, preferably for a method described herein. In an embodiment, the dsDNA comprises a dsDNA region at least 45 base pairs in length.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIG. 1 illustrates a change in body weight of mice implanted with B16-F10 (melanoma model) and treated with DNA-LNP. DNA-LNP refers to lipid nanoparticle (LNP) containing dsDNA. ADU-S100 is a stimulator of interferon genes (STING) agonist.

FIG. 2 illustrates tumor growth in mice implanted with B16-F10 and treated with DNA-LNP. The p-values are t-test significance compared to glucose/PBS at day 17: * indicates a P-value<0.05, ** indicates a P-value<0.01.

FIG. 3 illustrates change in body weight of mice implanted with B16-F10 (lung cancer model) and treated with DNA-LNP.

FIG. 4A, FIG. 4B, and FIG. 4C, illustrate serum cytokines in mice implanted with B16-F10 and treated with DNA-LNP. FIG. 4A shows IFN-β, FIG. 4B shows IL-6, and FIG. 4C shows IL-1β.

FIG. 5A and FIG. 5B illustrates lung weight and tumor nodule number in mice implanted with B16-F10 and treated with DNA-LNP. Reference to “CR” indicates number complete response (tumor nodules). In group 3, one out of eight mice had a complete response. In the other groups, no mice had a complete response.

FIG. 6 illustrates increased survival in a mouse model of hepatocellular carcinoma (HCC). HCC was induced by administering to mice a plasmid expressing the transgene of the protooncogene MET and a plasmid expressing the transgene for CTNNB1. MET encodes the receptor protein kinase Met (MET). CTNNB1 encodes β-catenin. For high dose DNA-LNP, the percent survival after 120 days was 100%. For low dose DNA-LNP, the percent survival after 120 days was 80%. ** indicates a P-value<0.01 by a log-rank test.

FIGS. 7A, 7B, 7C, and 7D, illustrate the ability of DNA-LNP to induce antitumor efficacy as measured by oncogene end point expression in a liver cancer model. Liver cancer was induced by administering to mice a plasmid expressing the transgene of the protooncogene cMET and a plasmid expressing β-catenin. FIG. 7A shows relative levels in β-catenin mRNA measured by RT-PCT at day 12 (D12). FIG. 7B shows relative levels in β-catenin mRNA measured by RT-PCT at the endpoint. FIG. 7C shows relative levels in cMET measured by RT-PCT at day 12 (D12). FIG. 7D shows relative levels in cMET mRNA measured by RT-PCT at the endpoint. In the FIGS. 7A, 7B, 7C, and 7D the y-axis ΔΔCt indicates that measured mRNA levels are normalized to an internal housekeeping gene (Actinb) and to the Met+Cat vehicle group.

FIG. 8 is a graph illustrating tumor growth in mice implanted subcutaneously with B16-F10 cells on both flanks and treated with a DNA-LNP or ADU-S100 at one flank. Local tumor refers to the tumor injected with treatments. Distal tumor refers to the untreated tumor at the other flank. Noncoding plasmid and mouse IL-18 (mIL-18) plasmid are dsDNA, with different lengths, without and with a transgene expression cassette, respectively. The p values are t-test significance compared to the PBS/Glucose (negative control) group on day 9: ** indicates a P-value<0.01, *** indicates a P-value<0.001, **** indicates a P-value<0.0001.

FIG. 9 is a graph showing survival of AML model mice following administration of DNA-LNP or ADU-S100. The p-values are log-rank test compared to vehicle: ** indicates a P-value<0.01; **** indicates a P-value<0.0001.

FIG. 10 is a graph showing survival of AML model mice following administration of DNA-LNP. LNP indicates “empty” LNP (no DNA). The p-values are log-rank test compared to vehicle: *** indicates a P-value<0.001; indicates a P-value<0.0001.

FIG. 11 illustrates the survival of AML model mice with depleted CD8+ cells following administration of the DNA-LNP. 200 μg of anti-CD8 was administrated intraperitoneally at d-2, 1, 4, and 7. *** indicates a P-value<0.001 by log-rank test compared to vehicle.

FIG. 12 is a graph showing survival of AML model mice after administration of 5 μg of DNA-LNP or DNA-LNP2 administration. LNP and LNP2 provide a different lipid nanoparticle composition. *** indicates a P-value<0.001 by log-rank test compared to vehicle.

FIG. 13 is a graph showing increased survival in AML model mice after administration of DNA-LNP or DNA2-LNP. DNA-LNP and DNA2-LNP provide for different DNA. The p-values are log-rank test compared to vehicle: *** indicates a P-value<0.001; **** indicates a P-value<0.0001.

FIG. 14 illustrates the number of CD8+ cells in the lung isolated from mice with B16-F10 (lung metastasis model) and treated with DNA-LNP or ADU-S100. Administration was given intravenously three times, three days apart starting 5 days after tumor cell injection. Lung tissue was collected 18 days after tumor cell injection and Formalin-fixed paraffin-embedded (FFPE) for immunohistochemistry (IHC) of CD8. Areas were annotated by hand in a blinded fashion. ** indicates a P-value<0.01 by t-test.

FIG. 15A, FIG. 15B, FIG. 15C, and FIG. 15D illustrate a dose-dependent increase of tumor infiltrating lymphocytes (TILs) due to DNA-LNP administration in the B16-F10 melanoma model as measured in CD45+ cells, CD8+ T cells, NK cells and a CD4:CD8 T cell ratio. The p values are t-test significance: * indicates a P-value<0.05; ** indicates a P-value<0.01; *** indicates a P-value<0.01.

FIG. 16A, FIG. 16B, FIG. 16C, and FIG. 16D illustrate a dose-dependent increase of activation markers on CD8+ T cells and NK cells due to DNA-LNP administration in the B16-F10 melanoma model. The p values are t-test significance: * indicates a P-value<0.05; ** indicates a P-value<0.01; *** indicates a P-value<0.01; **** indicates a P-value<0.0001.

FIG. 17A and FIG. 17B illustrate the survival of the DNA-LNP-dosed B16-F10 melanoma model after depleting CD8+ cells or NK cells. Groups “vehicle” and “DNA-LNP” were treated with IgG isotype. 200 μg of antibodies were dosed at d-2, 1, 4, 7, 10 μg of DNA-LNP was dosed at d0, 3, 6. * indicates a P-value<0.05 by log-rank test.

FIG. 18A and FIG. 18B illustrate tumor growth in mice that were implanted with B16-F10 and treated with anti-PD-L1 antibody. DNA-LNP, or a combination of anti-PD-L1 antibody and DNA-LNP. DNA-LNP was dosed at 1 μg (FIG. 18A) or 5 μg (FIG. 18B). Mice were dosed at d0, 3, 6. ** indicates a P-value<0.01 by t-test at d9.

FIG. 19A and FIG. 19B illustrates the combination effect of DNA-LNP and anti-PD-L1 on the survival of the B16-F10 melanoma model. Antibody or DNA-LNP were dosed at d0, 3, 6. The p-values are log-rank test compared to vehicle: * indicates a P-value<0.05; ** indicates a P-value<0.01.

FIGS. 20A-20E illustrate tumor growth in the HCC mouse model after DNA-LNP administration as measured by the luciferase activity in serum. LNP indicates “empty” LNP (no DNA). FIG. 20A illustrates results with vehicle. FIG. 20B illustrates results with LNP. FIG. 20C illustrates results with DNA-LNP (1 μg). FIG. 20D illustrates results with DNA-LNP (0.2 μg). FIG. 20E illustrates results with DNA-LNP (0.2 μg×3) three times, one week apart.

FIG. 21 illustrates the survival of the HCC mouse model after DNA-LNP or “empty” LNP (no DNA) administration. * indicates a P-value<0.05 by log-rank test compared to the “empty” LNP-dosed group.

FIG. 22 illustrates the change in body weight of the HCC mouse model a day after DNA-LNP or “empty” LNP (no DNA) administration.

### DETAILED DESCRIPTION OF THE INVENTION

The present invention features methods utilizing nanoparticles for delivering a dsDNA. As illustrated in the Examples below, the dsDNA can inhibit different cancers including lung, melanoma, liver and leukemia. While not wishing to be bound by any theory, the nanoparticles facilitate intracellular delivery of the dsDNA, where the dsDNA stimulates the innate immune response of the cytosolic sensing cGAS/STING and/or inflammasome pathways to provide an immune response. The Examples provided below include examples illustrating the effect of nanoparticle delivered dsDNA on cytokines, tumor size, and/or survival. The employed models include liver, lung, leukemia, melanoma, and checkpoint inhibitor resistant melanoma. In contrast to the STING agonist ADU-S100, dsDNA-LNP induced IL-18 and IFNγ, indicating activation of the inflammasome pathway (IL-18) by dsDNA-LNP may contribute to induce IFNγ production. In the checkpoint resistant melanoma study, anti-PDL1 antibody had no effect on tumor growth, while a synergistic effect was seen with dsDNA-LNP+anti-PDL1 antibody.

Method for treating cancer may involve one or more additional therapeutics agents having anti-cancer activity. The particular agent can, for example, directly target the cancer and/or can, for example, be an immunomodulator such as a checkpoint inhibitor.

Reference to “dsDNA” provides one or more polynucleotides that forms one or more double-stranded DNA regions. A dsDNA region may be formed from a single polynucleotide or two different polynucleotides. dsDNA may contain modified nucleotides, for example, sugar modifications (e.g., 2′-methoxyethyl (2′-MOE), 2′-fluor (2′-F), locked nucleic acid (LNA), constrained ethyl (cEt) and tricyclo-DNA (tc-DNA)), base modifications (e.g., C7-modified deaza-adenine (e.g., methyl, Cl or F), C7-modified deaza-guanosine (e.g., methyl, Cl or F), C5-modified cytosine (e.g., methyl, F or Cl), and C5-modified uridine (e.g., methyl, F or Cl)), and/or backbone modifications (e.g., phosphorothioate (Rp and/or Rs), thio-phosphoramidate, phosphorodiamidate morpholino oligos (PMO), and peptide-nucleic acid (PNA)). Examples of modified nucleotides are provided in, for example, Adachi et al., (2021) Biomedicines 9, 550; Shen and Corey (2018) Nucleic Acid Research 46:4, 1584-1600; and Duffy et al., (2020) 18:112; each of with are hereby incorporated by reference herein in their entirety.

Reference to “polynucleotide” provides a nucleic acid polymer made up of naturally occurring nucleotides and/or modified nucleotides. Nucleotides may contain sugar modifications (e.g., 2′-methoxyethyl (2′-MOE), 2′-fluor (2′-F), locked nucleic acid (LNA), constrained ethyl (cEt) and tricyclo-DNA (tc-DNA)), base modifications (e.g., C7-modified deaza-adenine (e.g., methyl, Cl or F), C7-modified deaza-guanosine (e.g., methyl, Cl or F), C5-modified cytosine (e.g., methyl, F or Cl), and C5-modified uridine (e.g., methyl, F or Cl)), and/or backbone modifications (e.g., phosphorothioate (Rp and/or Rs), thio-phosphoramidate, phosphorodiamidate morpholino oligos (PMO), and peptide-nucleic acid (PNA)).

dsDNA comprises a dsDNA region and may also comprise additional regions. Examples of additional regions include single-stranded regions, RNA regions, modified RNA regions, modified DNA regions and regions that are not nucleotides. In certain embodiments the dsDNA comprises a continuous polynucleotide strand providing a structure with a dsDNA region (e.g., a hair-pin loop) or comprises two polynucleotide strands where all or a region of the two strands form the dsDNA region. In different embodiments, the dsDNA is a minicircle, a nanoplasmid, open linear duplex DNA, or closed-ended linear duplex DNA (CELiD/ceDNA/doggybone DNA).

dsDNA can be produced using different techniques including enzymatic production of nucleotide polymers and/or chemical modification. Examples of techniques for producing nucleic acid are well known in the art and include, for example: Kosuri et al., (2014) Nat. Methods. 11 (5): 499-507; Ducani et al., (2013) Nat. Methods 10, 647-652; Ducani et al., (2014) Nucleic Acids Research, Volume 42, Issue 16; and Sandahl et al., (2021) Nat. Commun. 12, 2760.

In certain embodiments, nucleotide modifications do not significantly decrease the ability of the dsDNA to stimulate the innate immune response. In different embodiments, the dsDNA is able to stimulate an innate immune response of at least 50%, at least 65%, at least 75%, at least 85%, at least 90%, or least 100% compared to the corresponding unmodified dsDNA as measured by IFN-β, IL-6 and/or IL-1β as provided in the Examples below.

A “nanoparticle” refers to a small non-viral particle that can encapsulate or associate with dsDNA and facilitates dsDNA delivery to a cell. Examples of nanoparticles include lipid nanoparticles (LNP), polymeric nanoparticles, lipid polymer nanoparticles (LPNP), protein and peptide-based nanoparticles, DNA dendrimers and DNA-based nanocarriers, carbon nanotubes, microparticles, microcapsules, inorganic nanoparticles, peptide cage nanoparticles, and exosomes. The nanoparticle range in size from about 10 nm to about 1000 nm. In different embodiments, the nanoparticle is about 50 nm to about 500 nm, or about 50 nm to about 200 nm.

Reference to “subject” indicates a mammal, including humans; non-human primates such as apes, gibbons, gorillas, chimpanzees, orangutans, macaques; domestic animals, such as dogs and cats; farm animals such as poultry and ducks, horses, cows, goats, sheep, and pigs; and experimental animals such as mice, rats, rabbits, and guinea pigs. A preferred subject is a human subject.

A DNA vector contains a transgene operative linked to one or more regulatory element providing for RNA expression from the transgene. The produced RNA can itself be functional or can encode for a protein. One type of regulatory element is a promoter, which binds RNA polymerase and the necessary transcription factors to initiate transcription. When encoding for protein, the produced RNA sequence will also encode a termination sequence at the end of the coding sequence.

The term “operatively linked” refers to the association of two or more nucleic acid segments on a single nucleic acid where the function of one is affected by the other.

Reference to “transgene” indicates a DNA region capable of being expressed to RNA, without regard to origin of the polynucleotide sequence. The transgene is generally part of a longer length nucleic acid, where the nucleic acid contains at least one region with which the transgene is not normally associated with in nature.

The singular forms “a,” “an,” and “the” include plural reference unless the context clearly dictates otherwise.

As used herein, the conjunctive term “and/or” between multiple recited elements is understood to encompass both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning, and therefore satisfy the requirement of the term “and/or”. Concurrent applicability of more than one of the options is also understood to fall within the meaning of the term “and/or.”

Unless clearly indicated otherwise by the context employed, the terms “or” and “and” have the same meaning as “and/or”.

Reference to terms such as “including”, “for example”, “e.g.,”, “such as” followed by different members or examples, are open-ended descriptions where the listed members or examples are illustrative and other member or examples can be provided or used.

The terms “polypeptides,” “proteins” and “peptides” can be used interchangeably to refer to an amino acid sequence without regard to function. Polypeptides and peptides contain at least two amino acids, while proteins contain at least about 50 amino acid acids. The provided amino acids include naturally occurring amino acids and modified amino acids such as those provided by cellular modification.

Reference to “comprise”, and variations such as “comprises” and “comprising”, used with respect to an element or group of elements is open-ended and does not exclude additional unrecited elements or method steps. Terms such as “including”, “containing” and “characterized by” are synonymous with comprising. In the different aspects and embodiments described herein, reference to an open-ended term such as “comprising” can be replaced by the terms “consisting” or “consisting essentially of”.

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

The term “about” refers to a value within 10% of the underlying parameter (i.e., plus or minus 10%). For example, “about 1:10” includes 1.1:10.1 or 0.9:9.9, and “about 5 hours” includes 4.5 hours or 5.5 hours. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100%, as well as 95.1%, 95.2%, 95.3%, 95.4%, 95.5%, etc., 96.1%, 96.2%, 96.3%, 96.4%, 96.5% and so forth; reference to a numerical range, such as “1-4” includes 2, 3, as well as 1.1, 1.2, 1.3, 1.4 and so forth; reference to “1 to 4 weeks” includes 7, 8, 9, 10, 11, 12, 13, 14, 15, 16, 17, 18, 19, 20, 21, 22, 23, 24, 25, 26, 27, or 28 days.

Reference to an integer with more (greater) or less than includes numbers greater or less than the reference number, respectively. Thus, for example, reference to more than 2 includes 3, 4, 5, 6, 7, 8, 9, 10, 11, 12, 13, 14, 15; and administration “two or more” times includes 2, 3, 4, 5, 6, 7, 8, 9, 10, 11, 12, 13, 14, 15, or more times.

Various references including articles and patent publications are cited or described in the background and throughout the specification. Each of these references is herein incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section and other sections of the application apply throughout the present application.

Unless defined otherwise, all technical and scientific terms used herein have the same meaning commonly understood to one of ordinary skill in the art to which this invention pertains.

The description has been separated into various sections and paragraphs, and provides various embodiments. These separations should not be considered as disconnecting the substance of a paragraph or section or embodiments from the substance of another paragraph or section or embodiment. The provided descriptions have broad application and encompasses all the combinations of the various sections, paragraphs and sentences that can be contemplated. The discussion of any embodiment is meant only to be exemplary and is not intended to suggest the scope of the disclosure, including the claims (unless otherwise provided in the claims), is limited to these examples.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, aspects that are not expressly excluded in the instant invention are nevertheless disclosed herein.

### I. Nanoparticles

A variety of different nanoparticles can be employed including lipid nanoparticles (LNP), polymeric nanoparticles, lipid polymer nanoparticles (LPNP), protein and peptide-based nanoparticles, DNA dendrimers and DNA-based nanocarriers, carbon nanotubes, microparticles, microcapsules, inorganic nanoparticles, peptide cage nanoparticles, and exosomes. (See, e.g., Riley and Vermerris Nanomaterials (2017) 201, 7, 94; Thomas et al., Molecules (2019), 24, 3744; Bochicchio et al., (2021), 13, 198; Munagala et al., Cancer Letters (2021), 505, 58; Fu et al., (2020) NanoImpact 20, 100261; and Neshat et al. (2020) Current Opin. Biotechnol. 66:1-10.)

If desired, a nanoparticle can target a cell type using, for example, targeting ligands recognizing a target cell receptor. Examples of targeting ligands include carbohydrates (e.g., galactose, mannose, glucose, and galactomannan), endogenous ligands (e.g., folic acid and transferrin), antibodies (e.g., anti-HER2 antibody and hD1) and protein/peptides (e.g., RGD, epidermal growth factor, and low density lipoprotein) and peptides. (For example, Teo et al., Advanced Drug Delivery Reviews (2016), 98, 41.)

The present application features the use of nanoparticles to deliver dsDNA. In different embodiments, nanoparticles can deliver additional therapeutic compounds; one or more additional compounds is provided in different nanoparticles; and one or more additional compounds is provided in the same nanoparticle as the dsDNA. Reference to compound includes small molecules and large molecules (e.g., therapeutic proteins and antibodies).

The production of different nanoparticles and incorporation of nucleic acid and other compounds is well known in the art, and exemplified by different publications throughout the discussion in Section I. Examples of publications illustrating incorporation of nucleic acid in a particular nanoparticle such as an LPNP and a LNP include Teo et al., Advanced Drug Delivery Reviews (2016) 98, 41; Bochicchio et al., Pharmaceutics (2021) 13, 198; Mahzabin and Das, IJPSR (2021) 12 (1), 65; and Teixeira et al., (2017) Prog. Lipid Res. October; 68:1-11 (each of which are hereby incorporated by reference herein in their entirety). Factors that may impact small molecule incorporation into a nanoparticle include hydrophobicity and the presence of an ionizable moiety. (See, e.g., Nii and Ishii International Journal of Pharmaceutics (2005) 298, 198; and Chen et al., Journal of Controlled Release (2018) 286, 46.)

### I. A. Lipid-Based Delivery Systems

Lipid-based delivery systems include the use of a lipid as a component. Examples of lipid-based delivery systems include liposomes, lipid nanoparticles, micelles, and extracellular vesicles. In certain embodiments, the lipid nanoparticle comprises one or more internal ordered lipid structures, as opposed to, for example a liposome that comprises a complete lipid bilayer and an aqueous core.

A “lipid nanoparticle” or “LNP” refers to a lipid-based vesicle useful for delivery of nucleic acid molecules and having dimensions on the nanoscale. In different embodiments, the nanoparticle is from about 10 nm to about 1000 nm, about 50 nm to about 500 nm, or about 50 nm to about 200 nm.

DNA is negatively charged. Thus, it can be beneficial for the LNP to comprise a cationic lipid such as, for example, an amino lipid. Exemplary amino lipids are described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, all of which are incorporated herein in their entirety. In certain embodiments, the LNP comprises amino lipids such as any of those described in WO2013/063468, hereby incorporated herein in its entirety.

The terms “cationic lipid” and “amino lipid” are used interchangeably herein to include lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipid can also be titratable cationic lipids. In certain embodiments, the cationic lipids comprise a protonatable tertiary amine (e.g., pH-titratable) group; C18 alkyl chains, wherein each alkyl chain independently can have one or more double bonds, one or more triple bonds; and ether, ester, or ketal linkages between the head group and alkyl chains.

Cationic lipids include 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino) butanoate (DLin-M-C3-DMA, also known as MC3), salts thereof, and mixtures thereof. Other cationic lipids also include 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, Y-DLen-C2K-DMA, and (DLin-MP-DMA) (also known as 1-B11).

Still further cationic lipids include 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DMA), N,N-dioleyl-N,N-dimethylammonium chloride (DODAC), N-(1-(2,3-dioleyloxy) propyl)-N,N,N-trimethylammonium chloride (DOTMA), N,N-distearyl-N,N-dimethylammonium bromide (DDAB), N-(1-(2,3-dioleoyloxy) propyl)-N,N,N-trimethylammonium chloride (DOTAP), 3-(N—(N′,N′-dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

Additional ionizable lipids that can be used include C12-200, 3060i10, MC3, CKK-E12, Lipid 5, Lipid 9, ATX-002, ATX-003, and Merck-32. US Patent Publication No. 2017/0367988, describes Merck-32.

In further embodiments, cationic lipid can be present in an amount from about 10% by molar ratio of the LNP to about 85% by molar ratio of the LNP, or from about 50% by molar ratio of the LNP to about 75% by molar ratio of the LNP.

LNP can comprise a neutral lipid. Neutral lipids can comprise lipid species existing either in an uncharged or neutral zwitterionic form at physiological pH. Such lipids include diacylphosphatidylcholine, diacylphosphatidylethanolamine, ceramide, sphingomyelin, dihydrosphingomyelin, cephalin, and cerebrosides. The selection of neutral lipids is generally guided by considerations including particle size and stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized. In certain embodiments, lipids containing saturated fatty acids with carbon chain lengths in the range of C14 to C22 can be used. In certain embodiments lipids with mono or di-unsaturated fatty acids with carbon chain lengths in the range of C14 to C22 are used. Additionally, lipids having mixtures of saturated and unsaturated fatty acid chains can be used. Exemplary neutral lipids include 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or a phosphatidylcholine. The neutral lipids can also be composed of sphingomyelin, dihydrosphingomyelin, or phospholipids with other head groups, such as serine and inositol.

In further embodiments, providing for neutral lipids, the neutral lipid can be present in an amount from about 0.1% by weight of the lipid nanoparticle to about 99% by weight of the LNP, or from about 5% by weight of the LNP to about 15% by weight of the LNP, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

LNP can be combined with additional components such as sterols and polyethylene glycol. Sterols can confer fluidity to the LNP. As used herein “sterol” refers to naturally occurring sterol of plant (phytosterols) or animal (zoosterols) origin as well as non-naturally occurring synthetic sterols, all of which are characterized by the presence of a hydroxyl group at the 3-position of the steroid A-ring. Suitable sterols include those conventionally used in the field of liposome, lipid vesicle or lipid particle preparation, most commonly cholesterol. Phytosterols include campesterol, sitosterol, and stigmasterol. Sterols also include sterol-modified lipids, such as those described in U.S. Patent Application Publication 2011/0177156. In different embodiments providing for a sterol, the sterol is present in an amount from about 1% by weight of the LNP to about 80% by weight of the LNP or from about 10% by weight of the LNP to about 25% by weight of the LNP.

Polyethylene glycol (PEG) is a linear, water-soluble polymer of ethylene PEG repeating units with two terminal hydroxyl groups. PEGs are classified by their molecular weights, for example, PEG 2000 has an average molecular weight of about 2,000 daltons, and PEG 5000 has an average molecular weight of about 5,000 daltons. PEGs commercially available from Sigma Chemical Co. and other companies and include monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments concerning PEG, PEG has an average molecular weight of about 550 to about 10,000 daltons and is optionally substituted by alkyl, alkoxy, acyl or aryl. In further embodiments, the PEG can be substituted with methyl at the terminal hydroxyl position. In further embodiments, the PEG can have an average molecular weight from about 750 to about 5,000 daltons, or from about 1,000 to about 5,000 daltons, or from about 1,500 to about 3,000 daltons or from about 2,000 daltons or from about 750 daltons.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines.

In certain embodiments, the PEG-modified lipid can be PEG-modified diacylglycerols and dialkylglycerols. In certain embodiments, the PEG can be in an amount from about 0.1% by weight of the LNP to about 50% by weight of the LNP, or from about 5% by weight of the LNP to about 15% by weight of the LNP.

In further embodiments, concerning LNP size, prior to encapsulating, LNPs can have a size in a range from about 10 nm to 500 nm, or from about 50 nm to about 200 nm, or from 75 nm to about 125 nm.

In certain embodiments concerning LNP, the LNP is described by Billingsley et al., Nano Lett. 2020, 20, 1578 or Billingsley et al., International Patent Publication No. WO 2021/077066 (both of which are hereby incorporated by reference herein in their entirety). Billingsley et al., and WO2021/077066 describe LNPs containing lipid-anchored PEG, cholesterol, phospholipid and ionizable lipids. In certain embodiments, the LNP contains a C14-4 polyamine core and/or has a particle size of about 70 nm. C14-4 has the following structure.

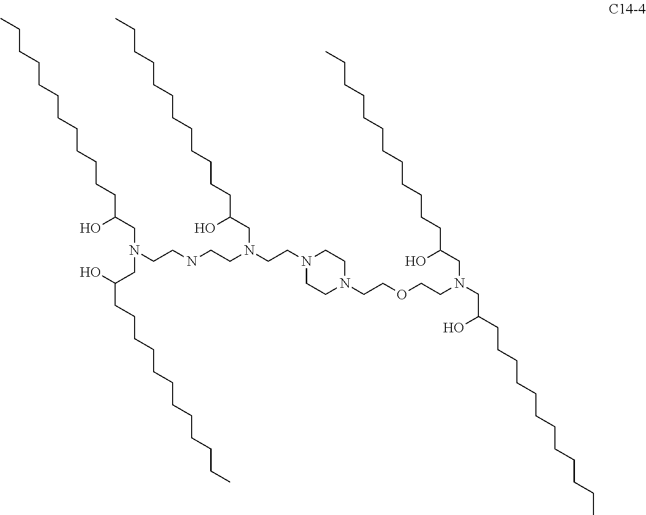

In certain embodiments the LNP is made up of a cationic lipid or lipopeptide described by U.S. Pat. Nos. 10,493,031, 10,682,374 or WO2021/077066 (each of which is hereby incorporated by reference herein in its entirety). In certain embodiments, the LNP contains a cationic lipid, a cholesterol-based lipid, and/or one or more PEG-modified lipids. In certain embodiments the LNP contains cKK-E12 (Dong et al., PNAS (2014) 111 (11), 3955):

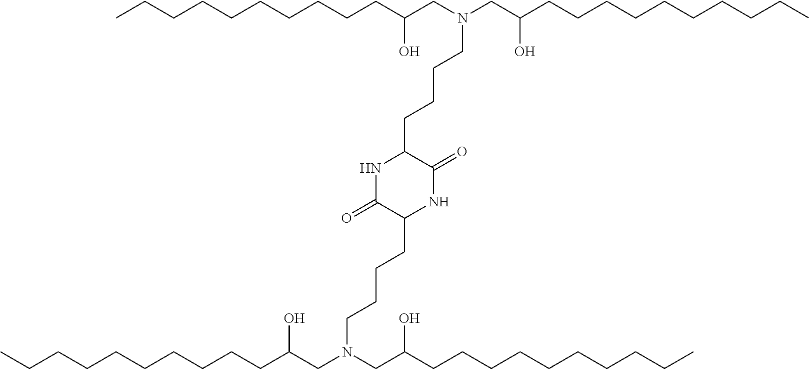 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In certain embodiments the LNP comprises a modified form of cKK-E12 referred to herein as “bCKK-E12,” having the following structure:

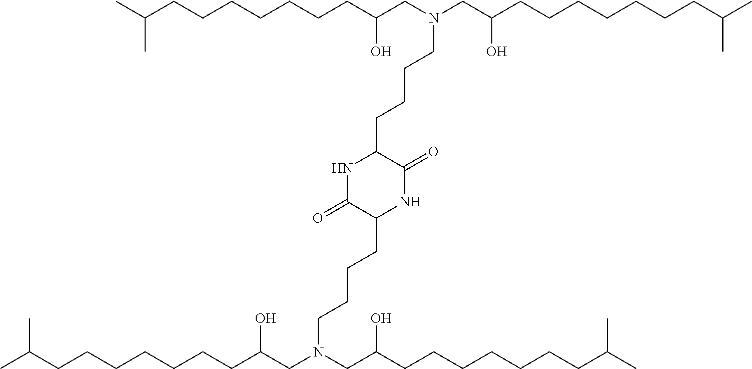 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

In certain embodiments the LNP comprises Lipid 1, 2, 3, 4, 5, 6, 7, 8, 9, or 10 as described by Sabnis et al., Molecular Therapy 2018, 26:6, 1509-1519 (hereby incorporated by reference herein in its entirety). In certain embodiments the LNP comprises Lipid 5, 8, 9, 10, or 11 described in Sabnis et al.

Lipid 5 of Sabnis et al. has the structure:

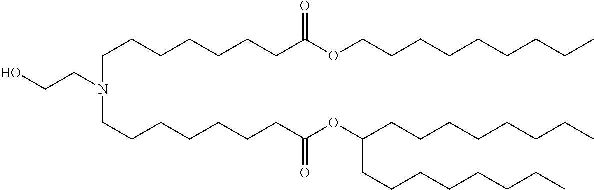 [SMILES: CCCCCCCCCOC(=O)CCCCCCCN(CCO)CCCCCCCC(=O)OC(CCCCCCCC)CCCCCCCC]

Lipid 9 of Sabnis et al. has the structure:

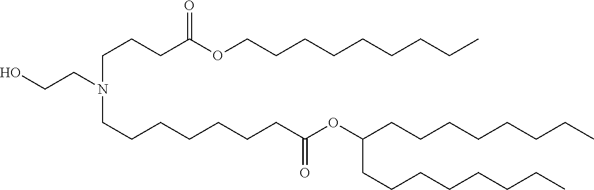 [SMILES: CCCCCCCCCOC(=O)CCCN(CCO)CCCCCCCC(=O)OC(CCCCCCCC)CCCCCCCC]

Additional lipids that may be utilized include those described by Roces et al., Pharmaceutics, 2020, 12,1095; Jayaraman et al, Angew. Chem. Int. Ed., 2012, 51, 8529-8533; Maier et al., www.moleculartherapy.org, 2013, Vol. 21, No. 8, 1570-1578; Liu et al., Adv. Mater. 2019, 31, 1902575, e.g., BAMEA-O16B; Cheng et al., Adv. Mater., 2018, 30, 1805308, e.g., 5A2-SC8; Hajj and Ball, Small, 2019 15, 1805097, e.g., 3060i10; Du et al., U.S. Patent Application Publication No. 2016/0376224; and Tanaka et al., Adv. Funct. Mater., 2020, 30, 1910575; each of which are hereby incorporated by reference herein in their entirety.

In certain embodiments, the LNP comprises mol % of the following components: one or more cationic lipids from about 20% to 65%, one or more phospholipid lipids from about 1% to about 50%, one or more PEG-conjugated lipid from about 0.1% to 10%, and cholesterol from about 0% to about 70%; one or more cationic lipids from about 20% to 50%, one or more phospholipid lipids from about 5% to about 20%, one or more PEG-conjugated lipid from about 0.1% to 5%, and cholesterol from about 20% to about 60%; in additional embodiments the phospholipid lipid is a neutral lipid; and the phospholipid lipid is DOPE or DSPC.

In certain embodiments the LNP, in mole %, comprises, consists essentially, or consists of the following components: (1) cKK-E12 (further described above and in Dong et al., PNAS (2014) 111 (11), 3955), about 35%; C14-PEG2000, about 2.5%; cholesterol, about 46.5%; and DOPE, about 16%; or (2) Lipid 9 (Lipid 9 further described above and in Sabnis et al., (2018) Molecular Therapy 26:6, 1509-1519), about 50%; C14-PEG2000, about 1.5%; cholesterol, about 38.5%; and DSPC, about 10%.

In certain embodiment, the LNP, in mole %, comprises, consists essentially, or consists of the following components: bCKK-E12, about 35%; C14-PEG2000, about 2.5%; cholesterol, about 46.5%; and dioleoylphosphatidylethanolamine (DOPE), about 16%. Additional aspects and embodiments include:

1. A lipid nanoparticle comprising a double-stranded DNA (dsDNA), wherein said dsDNA comprises a double-stranded region of at least 45 base pairs in length and said dsDNA is non-coding or lacks a promoter operatively linked to a region coding for expression in said subject.

2. The lipid nanoparticle of 1, wherein said lipid nanoparticle comprises mol % one or more cationic lipids from about 20% to about 65%, one or more phospholipids from about 1% to about 50%, one or more PEG-conjugated lipids from about 0.1% to about 10%, and cholesterol from about 0% to about 70%.

3. The lipid nanoparticle of 1, wherein said lipid nanoparticle comprises mol % one or more cationic lipids from about 20% to about 50%, one or more phospholipids from about 5% to about 20%, one or more PEG-conjugated lipids from about 0.1% to about 5%, and cholesterol from about 20% to about 60%.

4. The lipid nanoparticle of 2 or 3, wherein said phospholipid lipid is 1,2-dioleoyl-sn-glycero-3-phosphatidyl-ethanolamine or 1,2-distearoyl-sn-glycero-3-phosphocholine.

5. The lipid nanoparticle of 1, wherein said lipid nanoparticle in mole %, comprises the following components (1) cKK-E12, about 35%; C14-PEG2000, about 2.5%; cholesterol, about 46.5%; and 1,2-dioleoyl-sn-glycero-3-phosphatidyl-ethanolamine (DOPE), about 16%; or (2) Lipid 9, about 50%; C14-PEG2000, about 1.5%; cholesterol, about 38.5%; and 1,2-distearoyl-sn-glycero-3-phosphocholine (DSPC), about 10%.

6. The lipid nanoparticle of any of 1-5, wherein said dsDNA region is at least 200 base pairs in length.

7. The lipid nanoparticle of any one of 1-6, wherein said dsDNA is unmodified.

8. The lipid nanoparticle of any one of 1-7, wherein said dsDNA is linear or circular.

9. The lipid nanoparticle of any one of claims 1-7, wherein said dsDNA is selected from the group consisting of a minicircle, a plasmid, an open linear duplex DNA, and a closed-ended linear duplex DNA.

10. The lipid nanoparticle of any one of 1-7, wherein said dsDNA region is provided by two regions of a polynucleotide and said polypeptide comprises a loop region.

### I.B. Polymer-Based Nanoparticles

Polymer-based delivery systems can be made from a variety of different natural and synthetic materials. DNA and other compounds can be entrapped into the polymeric matrix of polymeric nanoparticles or can be adsorbed or conjugated on the surface of the nanoparticles. Examples of commonly used polymers for nucleic acid delivery include poly(lactic-co-glycolic acid) (PLGA), poly lactic acid (PLA), poly(ethylene imine) (PEI) and PEI derivatives, chitosan, dendrimers, polyanhydride, polycaprolactone, polymethacrylates, poly-L-lysine, pullulan, dextran, and hyaluronic acid, poly-b-aminoesters. (Thomas et al., (2019) Molecules 24, 3744.)

Polymeric-based nanoparticles can have different sizes, ranging from about 1 nm to about 1000 nm, from about 10 nm to about 500 nm, from about 50 nm to about 200 nm, from about 100 nm to about 150 nm, and from about 150 nm or less.

### I.C. Lipid Polymer Nanoparticles

Lipid polymer nanoparticles are hybrid nanoparticles providing both a lipid component and a polymer component, and as such can be considered to be an LNP or LPNP. The LPNP configuration can provide an outer polymer and inner lipid or an outer lipid and inner polymer. The presence of two different types of material facilitates designing nanoparticles to provide for delayed release of a component. Different lipid and polymer components can be selected taking into account the material be delivered. (For example, see Teo et al., Advanced Drug Delivery Reviews (2016) 98, 41; Bochicchio et al., Pharmaceutics (2021) 13, 198; Mahzabin and Das, IJPSR (2021) 12 (1), 65; and Teixeira et al., (2017) Prog. Lipid Res. October; 68:1-11.)

### I.D. Protein and Peptide-Based Nanoparticles

Protein and peptide-based systems can employ a variety of different proteins and peptides. Examples of proteins include gelatin and elastin. Peptide-based systems can employ, for example, CPPs.

CPPs are short peptides (6-30 amino acid residues) potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25 (1): 1996-2006). Examples of CPPs include cationic CPPs (highly positively charged) such as the Tat peptide, penetratin, protamine, poly-L-lysine, and polyarginine; amphipathic CPPs (chimeric or fused peptides, constructed from different sources, containing both positively and negatively charged amino acid sequences), such as transportan, VT5, bactenecin-7 (Bac7), proline-rich peptide (PPR), SAP (VRLPPP) 3, TP10, pep-1, and MPG); membranotropic CPPs (exhibit both hydrophobic and amphipathic nature simultaneously, and comprise both large aromatic residues and small residues) such as H625, SPIONs-PEG-CPP and NPs; and hydrophobic CPPs (contain only non-polar motifs or residues) such as SG3, PFVYLI, pep-7, and fibroblast growth factors.

The protein and peptide nanoparticles can be provided in different sizes, for example, ranging from about 1 nm to about 1000 nm, from about 10 nm to about 500 nm, from about 50 nm to about 200 nm, from about 100 nm to about 150 nm, or from about 150 nm or less.

### I.E. Peptide Cage Nanoparticles

Peptide cage-based delivery systems can be produced from proteinaceous material able to assemble into a cage-like structure forming a constrained internal environment. Peptide cages can comprise a proteinaceous shell that self-assembles to form a protein cage (e.g., a structure with an interior cavity that is either naturally accessible to the solvent or can be made so by altering solvent concentration, pH, or equilibria ratios). The monomers of the protein cages can be naturally occurring or variant forms, including amino acid substitutions, insertions, and deletions (e.g., fragments).

Different types of protein “shells” can be assembled and loaded with different types of materials. Protein cages can be produced using viral coat protein(s) (e.g., from the Cowpea Chlorotic Mottle Virus protein coat), as well non-viral proteins (e.g., U.S. Pat. Nos. 6,180,389 and 6,984,386, U.S. patent application No. 20040028694, and U.S. patent application Ser. No. 20/090,035389, each of which is incorporated by reference herein in their entity).

Examples of protein cages derived from non-viral proteins include: eukaryotic or prokaryotic derived ferritins and apoferritins such as 12 and 24 subunit ferritins; and heat shock proteins (HSPs), such as the class of 24 subunit heat shock proteins that form an internal core space, the small HSP of Methanococcus jannaschii, the dodecameric Dsp HSP of E. coli; and the MrgA protein.

Protein cages can have different core sizes, such as ranging from about 1 nm to about 1000 nm, from about 10 nm to about 500 nm, from about 50 nm to about 200 nm, from about 100 nm to about 150 nm, or from about 150 nm or less.

### I.F. Exosomes

Exosomes are small biological membrane vesicles that been utilized to deliver various cargoes including small molecules, peptides, proteins and nucleic acids. Exosomes generally range in size from about 30 nm to 100 nm and can be taken up by a cell and deliver its cargo. Cargoes can be associated with exosome surface structure or may be encapsulated within the exosome bilayer.

Various modifications can be made to exosomes facilitating cargo delivery and cell targeting. Modifications for facilitating cargo delivery include structures for associating with cargoes such as protein scaffolds and polymers. Modifications for cell targeting include targeting ligands and modifying surface charge. Publications describing production, modification, and use of exosomes for delivery of different cargoes include Munagala et al., Cancer Letters (2021), 505, 58; Fu et al., (2020) NanoImpact 20, 100261; and Dooley et al., (2021) Molecular Therapy 29 (5), 1729 (each of which is hereby incorporated by reference herein).

### II. Cancer Treatment and Therapeutic Agents

Nanoparticle administration of dsDNA can be used to treat or enhance treatment of a variety of different cancers. Cancer is due to an abnormal uncontrolled cell growth that may result in an increase solid mass (tumor) or exist in the blood. Examples of cancers include those provided at: hypertext transfer protocal://www.cancer.gov/types. In certain embodiments, the cancer is a solid tumor. In a first group of cancers, the cancer is selected from the group consisting of: acute lymphoblastic leukemia (ALL); acute myeloid leukemia (AML); adrenocortical carcinoma; aids-related cancer (Kaposi sarcoma); aids-related lymphoma; primary CNS lymphoma; anal cancer; astrocytomas; atypical teratoid/rhabdoid tumor: central nervous system cancer; basal cell carcinoma of the skin; bile duct cancer; bladder cancer; bone cancer (e.g., Ewing Sarcoma and osteosarcoma and malignant fibrous histiocytoma); brain tumors; breast cancer; bronchial tumors (lung cancer); Burkitt lymphoma; carcinoid tumor (gastrointestinal); cardiac (heart) tumors; medulloblastoma and other CNS embryonal tumors; germ cell tumor, childhood (brain cancer); cervical cancer; cholangiocarcinoma; chordoma (bone cancer); chronic lymphocytic leukemia; chronic myelogenous leukemia; chronic myeloproliferative neoplasms; colorectal cancer; craniopharyngioma (brain cancer); lymphoma (e.g., mycosis fungoides and Sézary Syndrome); Ductal Carcinoma In Situ (DCIS); endometrial cancer (uterine cancer); ependymoma (brain cancer); esophageal cancer; esthesioneuroblastoma (head and neck cancer); extracranial germ cell tumor; extragonadal germ cell tumor; eye cancer (e.g., intraocular melanoma, and retinoblastoma); fallopian tube cancer; gallbladder cancer; gastric (stomach) cancer; gastrointestinal carcinoid tumor; gastrointestinal stromal tumors; germ cell tumors (e.g., childhood extracranial germ cell tumors, extragonadal germ cell tumors, ovarian germ cell tumors, testicular cancer, and gestational trophoblastic disease); hairy cell leukemia; head and neck cancer; hepatocellular (liver) cancer; Hodgkin lymphoma; hypopharyngeal cancer (head and neck cancer); intraocular melanoma; islet cell tumors, pancreatic neuroendocrine tumors; Kaposi Sarcoma; kidney (renal cell) cancer; Langerhans cell histiocytosis; laryngeal cancer (head and neck cancer); leukemia; lip and oral cavity cancer (head and neck cancer); lung cancer (non-small cell, small cell, pleuropulmonary blastoma, and tracheobronchial tumor); melanoma; Merkel cell carcinoma (skin cancer); mesothelioma; metastatic cancer; metastatic squamous neck cancer with occult primary (head and neck cancer); midline tract carcinoma with nut gene changes; mouth cancer (head and neck cancer); multiple endocrine neoplasia syndromes; multiple myeloma/plasma cell neoplasms; myelodysplastic syndromes; myelodysplastic/myeloproliferative neoplasms; myelogenous leukemia, chronic (CML); myeloproliferative neoplasms; nasal cavity and paranasal sinus cancer (head and neck cancer); nasopharyngeal cancer (head and neck cancer); neuroblastoma; non-small cell lung cancer; oral cancer, lip and oral cavity cancer and oropharyngeal cancer (head and neck cancer); osteosarcoma and undifferentiated pleomorphic sarcoma; ovarian cancer; pancreatic cancer; papillomatosis; paraganglioma; paranasal sinus and nasal cavity cancer (head and neck cancer); parathyroid cancer; penile cancer; pharyngeal cancer (head and neck cancer); pheochromocytoma; pituitary tumor; plasma cell neoplasm/multiple myeloma; pleuropulmonary blastoma (lung cancer); primary central nervous system (CNS) lymphoma; primary peritoneal cancer; prostate cancer; rectal cancer; retinoblastoma; rhabdomyosarcoma, childhood (soft tissue sarcoma); salivary gland cancer (head and neck cancer); sarcoma; childhood rhabdomyosarcoma (soft tissue sarcoma); childhood vascular tumors (soft tissue sarcoma); soft tissue sarcoma; uterine sarcoma; skin cancer; small cell lung cancer; small intestine cancer; soft tissue sarcoma: squamous neck cancer with occult primary, metastatic (head and neck cancer); stomach (gastric) cancer; t-cell lymphoma; testicular cancer; throat cancer (head and neck cancer); nasopharyngeal cancer; oropharyngeal cancer; hypopharyngeal cancer; thymoma and thymic carcinoma; thyroid cancer; tracheobronchial tumors (lung cancer); transitional cell cancer of the renal pelvis and ureter (kidney cancer); ureter and renal pelvis, transitional cell cancer (kidney cancer); urethral cancer; uterine cancer, endometrial; uterine sarcoma; vaginal cancer; vascular tumors; and vulvar cancer.

In a second group of cancers, the cancer is selected from the group consisting of: anal cancer, bladder cancer, bone cancer, brain tumors, breast cancer, cervical cancer, colon and rectal cancer, endometrial cancer, esophageal cancer, gastrointestinal stromal tumors, gestational trophoblastic disease, head and neck cancer, Hodgkin lymphoma, Kaposi sarcoma, kidney cancer, leukemia, liver cancer, lung cancer, malignant mesothelioma, melanoma, multicentric Castleman disease, multiple myeloma and other plasma cell neoplasms, myeloproliferative neoplasms, neuroblastoma, non-Hodgkin lymphoma, ovarian, fallopian tube, primary peritoneal cancer, pancreatic cancer, penile cancer, pheochromocytoma, paraganglioma, prostate cancer, retinoblastoma, rhabdomyosarcoma, skin cancer, soft tissue sarcoma, solid tumors anywhere in the body, stomach (gastric) cancer, testicular cancer, thyroid cancer, vaginal cancer, and vulvar cancer. Approved FDA drugs for treating these different cancers can accessed through hypertext transfer protocal://www.cancer.gov/types://www.cancer.gov/about-cancer/treatment/drugs/cancer-type (hereby incorporated by reference herein in its entirety).

In a third group of cancers, the cancer is selected from the group consisting of: bladder cancer, breast cancer, colon and rectal cancer, endometrial cancer, kidney cancer, leukemia, liver cancer, lung cancer, melanoma, non-Hodgkin lymphoma, pancreatic cancer, prostate cancer and thyroid cancer.

In a fourth group of cancers, the cancer is selected from the group consisting of: bladder cancer, breast cancer, colon and rectal cancer, endometrial cancer, kidney cancer, liver cancer, lung cancer, melanoma, pancreatic cancer, prostate cancer and thyroid cancer.

In a fifth group of cancers, the cancer is selected from the group consisting of liver cancer, lung cancer, and melanoma.

A variety of treatments can be employed to treat cancer including small molecules, proteins, antibodies, antibody fragments comprising an antigen binding region, antigen binding proteins, nanobodies, multi-specific antibodies, multi-specific antigen binding proteins and nucleic acids. Nucleic acid treatment may include providing a transgene encoding a cancer antigen, a therapeutic protein, antibody, antibody fragment comprising an antigen binding region, antigen binding protein, nanobody, multi-specific antibody, multi-specific antigen binding proteins or a functional nucleic acid targeting a cancer cell or cancer cell components; and oligodeoxynucleotides or oligonucleotides (ODNs) such as ODNs containing CpGs. Examples of functional nucleic acid targeting cancer cell nucleic acid include a short hair pin RNA (shRNA), a small interfering RNA (siRNA), a microRNA (miRNA), a RNAi, a ribozyme, an antisense RNA, a clustered regularly interspaced short palindromic repeats (CRISPR)/Cas9 construct, a zinc finger nuclease (ZFN), and or a transcription activator-like effector nuclease (TALEN). Examples of ODNs containing CpGs are provided in Hanagata International Journal of Nanomedicine (2012) 7:2181-2195; and Hanagata International Journal of Nanomedicine (2017) 12:515-531.

Examples of antibody fragments comprising an antigen binding region, antigen binding proteins, nanobodies, multi-specific antibodies (e.g., bispecific), multi-specific antigen binding proteins, that may be used in cancer treatment are provided in, for example, Yang et al., Front Oncol. (2020) 10:1182 and You et al., Vaccines (2021), 9, 724 (both of which are hereby incorporated by reference herein in their entirety). Reference to antigen binding regions indicate an antibody variable region able to bind to antigen.

Table 1 provides examples of approved FDA drugs (therapeutic agents) for treating melanoma, lung cancer and liver cancer. Such drugs can be used in combination with the nanoparticles comprising a dsDNA as provided for herein. Additional cancer therapeutics for different indications are known in the art and in development.

TABLE 1

| Cancer | |
|---|---|
| Type | FDA Approved drug |
| Melanoma | Aldesleukin, Binimetinib, Braftovi ® (Encorafenib), Cobimetinib Fumarate, |
| | Cotellic ® (Cobimetinib Fumarate), Dabrafenib Mesylate, Dacarbazine, |
| | Encorafenib, IL-2 (Aldesleukin), Imlygic (Talimogene Laherparepvec), |
| | Interleukin-2 (Aldesleukin), Intron A (Recombinant Interferon Alfa-2b), |
| | Ipilimumab, Keytruda ® (Pembrolizumab), Mekinist ® (Trametinib Dimethyl |
| | Sulfoxide), Mektovi ® (Binimetinib), Nivolumab, Opdivo ® (Nivolumab), |
| | Peginterferon Alfa-2b, PEG-Intron (Peginterferon Alfa-2b), Pembrolizumab, |
| | Proleukin ® (Aldesleukin), Recombinant Interferon Alfa-2b, Sylatron ® |
| | (Peginterferon Alfa-2b), Tafinlar ® (Dabrafenib Mesylate), Talimogene |
| | Laherparepvec, Trametinib Dimethyl Sulfoxide, Vemurafenib, Yervoy ® |
| | (Ipilimumab), and Zelboraf ® (Vemurafenib). |
| Non-Small Cell | Abraxane (Paclitaxel Albumin-stabilized Nanoparticle Formulation), Afatinib |
| Lung | Dimaleate, Afinitor (Everolimus), Afinitor Disperz (Everolimus), Alecensa |
| Cancer | (Alectinib), Alectinib, Alimta (Pemetrexed Disodium), Alunbrig (Brigatinib), |
| | Amivantamab-vmjw, Atezolizumab, Avastin (Bevacizumab), Bevacizumab, |
| | Brigatinib, Capmatinib Hydrochloride, Cemiplimab-rwlc, Ceritinib, Crizotinib, |
| | Cyramza (Ramucirumab), Dabrafenib Mesylate, Dacomitinib, Docetaxel, |
| | Doxorubicin Hydrochloride, Durvalumab, Entrectinib, Erlotinib Hydrochloride, |
| | Everolimus, Exkivity ® (Mobocertinib Succinate), Gavreto ® (Pralsetinib), |
| | Gefitinib ®, Gilotrif ® (Afatinib Dimaleate), Gemcitabine Hydrochloride, |
| | Gemzar ® (Gemcitabine Hydrochloride), Imfinzi ® (Durvalumab), Infugem ® |
| | (Gemcitabine Hydrochloride), Ipilimumab, Iressa ® (Gefitinib), Keytruda ® |
| | (Pembrolizumab), Libtayo ® (Cemiplimab-rwlc), Lorbrena ® (Lorlatinib), |
| | Lorlatinib, Lumakras ® (Sotorasib), Mekinist ® (Trametinib Dimethyl |
| | Sulfoxide), Methotrexate Sodium, Mobocertinib Succinate, Mvasi ® |
| | (Bevacizumab), Necitumumab, Nivolumab, Opdivo ® (Nivolumab), |
| | Osimertinib Mesylate, Paclitaxel, Paclitaxel Albumin-stabilized Nanoparticle |
| | Formulation, Pembrolizumab, Pemetrexed Disodium, Portrazza ® |
| | (Necitumumab), Pralsetinib, Ramucirumab, Retevmo ® (Selpercatinib), |
| | Rozlytrek ® (Entrectinib), Rybrevant ® (Amivantamab-vmjw), Selpercatinib, |
| | Sotorasib, Tabrecta ® (Capmatinib Hydrochloride), Tafinlar ® (Dabrafenib |
| | Mesylate), Tagrisso ® (Osimertinib Mesylate), Tarceva ® (Erlotinib |
| | Hydrochloride), Taxotere ® (Docetaxel), Tecentriq ® (Atezolizumab), |
| | Tepmetko ® (Tepotinib Hydrochloride), Tepotinib Hydrochloride, Trametinib |
| | Dimethyl Sulfoxide, Trexall ® (Methotrexate Sodium), Vizimpro ® |
| | (Dacomitinib), Vinorelbine Tartrate, Xalkori ® (Crizotinib), Yervoy ® |
| | (Ipilimumab), Zirabev ® (Bevacizumab), Zykadia ® (Ceritinib); Carboplatin- |
| | Taxol and Gemcitabine-Cisplatin |
| Small Cell | Afinitor ® (Everolimus), Atezolizumab, Doxorubicin Hydrochloride, |
| Lung | Durvalumab, Etopophos ® (Etoposide Phosphate), Etoposide, Etoposide |
| Cancer | Phosphate, Everolimus, Hycamtin ® (Topotecan Hydrochloride), Imfinzi ® |
| | (Durvalumab), Lurbinectedin, Methotrexate Sodium, Nivolumab, Opdivo ® |
| | (Nivolumab), Tecentriq ® (Atezolizumab), Topotecan Hydrochloride, Trexall ® |
| | (Methotrexate Sodium), and Zepzelca ® (Lurbinectedin) |
| Liver | Atezolizumab, Avastin ® (Bevacizumab), Bevacizumab, Cabometyx ® |
| Cancer | (Cabozantinib-S-Malate), Cabozantinib-S-Malate, Cyramza ® (Ramucirumab), |
| | Infigratinib Phosphate, Keytruda ® (Pembrolizumab), Lenvatinib Mesylate, |
| | Lenvima ® (Lenvatinib Mesylate), Nexavar ® (Sorafenib Tosylate), Nivolumab, |
| | Opdivo ® (Nivolumab), Pemazyre ® (Pemigatinib), Pembrolizumab, |
| | Pemigatinib, Ramucirumab, Regorafenib, Sorafenib Tosylate, Stivarga ® |
| | (Regorafenib), Tecentriq ® (Atezolizumab), and Truseltiq ® (Infigratinib |
| | Phosphate) |

Immunomodulators act on pathways that regulate the immune system and can be used to treat different disease including different types of cancers. One type of immunomodulators are checkpoint inhibitors. Certain proteins can act at immune checkpoints to decrease host immune responses. Checkpoint inhibitors block the ability of such proteins to decrease an immune response. A variety of different checkpoint inhibitors are in development and are FDA approved. Examples of FDA approved checkpoint inhibitors are provided in Table 2.

TABLE 2

| Antibody | Target | Approvals include |
|---|---|---|
| Atezolizumab (Tecentriq ®) | PD-L1 | subsets of patients with bladder cancer, |
| | | breast cancer, liver cancer, lung cancer, |
| | | and melanoma |
| Avelumab (Bavencio ®) | PD-L1 | subsets of patients with bladder cancer, |
| | | kidney cancer, and Merkel cell carcinoma, |
| | | a type of skin cancer |
| Cemiplimab (Libtayo ®) | PD-1 | subsets of patients with cutaneous |
| | | squamous cell carcinoma, basal cell |
| | | carcinoma, and lung cancer |
| Dostarlimab (Jemperli) | PD-1 | subsets of patients with uterine |
| | | (endometrial) cancer |
| Durvalumab (Imfinzi ™) | PD-L1 | subsets of patients with bladder cancer and |
| | | lung cancer |
| Ipilimumab (Yervoy ®) | CTLA-4 | subsets of patients with melanoma, |
| | | mesothelioma, liver cancer, and lung |
| | | cancer |
| Nivolumab (Opdivo ®) | PD-1 | subsets of patients with bladder cancer, |
| | | colorectal cancer, esophageal cancer, |
| | | gastric cancer, head and neck cancer, |
| | | kidney cancer, liver cancer, lung cancer, |
| | | lymphoma, melanoma, and mesothelioma |
| Pembrolizumab | PD-1 | subsets of patients with bladder cancer, |
| (Keytruda ®) | | breast cancer, cervical cancer, colorectal |
| | | cancer, cutaneous squamous cell |
| | | carcinoma, esophageal cancer, head and |
| | | neck cancer, kidney cancer, liver cancer, |
| | | lung cancer, lymphoma, melanoma, |
| | | Merkel cell carcinoma, and stomach |
| | | cancer; and cancers of any type that |
| | | present with certain genetic mutations |
| | | (MSI-H, dMMR, or TMB-H) |

Additional checkpoint inhibitors and indications are undergoing clinical trials. (See, for example, Darvin et al., Experimental & Molecular Medicine (2018) 50:165, hereby incorporated by reference herein in its entirety).

In certain embodiments a checkpoint inhibitor, preferably an inhibitor listed in Table 2, is used to treat cancer. In further embodiments, the cancer is stratified 13 or 14 based on quantification of CD3+ and CD8+ lymphocyte populations described in Pagès et al., (2018) Lancet 391:2128-2139, hereby incorporated by reference herein in its entirety.

In additional embodiments concerning the checkpoint inhibitor, the checkpoint inhibitor is an antibody targeting programmed cell death 1 receptor (PD-1) or programmed cell death receptor ligand 1 (PD-L1); the cancer is a tumor having PD-L1 expression on at least 1%, at least 5%, at least 10%, at least 20%, at least 25% or at least 50% of the tumor cells; and/or the checkpoint inhibitor is selected from the group consisting of: atezolizumab, avelumab, cemiplimab, dostarlimab, durvalumab, nivolumab, and pembrolizumab.

Nanoparticle delivery of dsDNA can be used in combination with a variety of different types of cancer vaccines. Examples of different types of cancer vaccines include peptide based vaccines, DNA and RNA vaccines, and whole cell vaccines. (Stephens et al., (2021) Frontiers in Immunology (2021) volume 12, article 696791; Paston et al., Frontiers in Immunology (2021) volume 12, article 627932; and Lopes et al., (2019) J. Exp. Clin. Cancer Res. (2019) 38, 146; each of which are hereby incorporated by reference herein in their entirety.)

Peptide based vaccines typically employ tumor specific or tumor associated antigen to produce an immune response targeting a cancer. Examples of tumor antigens include MAGE, NY-ESO-1, GAGE, BAGE, KRAS, p53, NRAS, BCR-ABL translocation, ETV6, NPM/ALK, ALK, EBV, LMP-1/LMP-2A, HPV E6/E7, HTLV-1, Tax, Melan, A/Mart-1, gp100, Tyrosinase, PSA, CEA, HER2, hTERT, Arginase-1, Survivin, MUC1, WT1, and cyclin B. A variety of different peptide based vaccines are in different stages of development including clinical trials. (Stephens et al., (2021) Frontiers in Immunology (2021) volume 12, article 696791 and Paston et al., Frontiers in Immunology (2021) volume 12, article 627932, both of which are incorporated by reference herein.)

DNA and RNA vaccines can be used to express vaccine antigens in a host. A variety of different nucleic acid vaccines are in different stages of development including clinical trials. (See, Lopes et al., (2019) J. Exp. Clin. Cancer Res. 38, 146 (2019), hereby incorporated by reference herein.)

In certain embodiments, the vaccine is (1) a peptide based vaccine, (2) a DNA vaccine or (3) an RNA vaccine: providing one or more such antigens selected from the group consisting of MAGE, NY-ESO-1, GAGE, BAGE, KRAS, p53, NRAS, BCR-ABL translocation, ETV6, NPM/ALK, ALK, EBV, LMP-1/LMP-2A, HPV E6/E7, HTLV-1, Tax, Melan, A/Mart-1, gp100, Tyrosinase, PSA, CEA, HER2, hTERT, Arginase-1, Survivin, MUC1, WT1, and cyclin B.

Cancer vaccines can further be combined with additional anti-cancer agents including small molecules and checkpoint inhibitors. In certain embodiments, the nanoparticle delivery of dsDNA is used with a cancer vaccine in combination with one or more therapeutic agent from Table 1 supra and/or one or more checkpoint inhibitor from Table 2 supra.

In certain embodiments treatment involves the use of a nanoparticle comprising dsDNA, in combination with a checkpoint inhibitor wherein: (i) the cancer is resistant to checkpoint inhibitor treatment, (ii) the subject has previously undergone a treatment with a checkpoint inhibitor in the absence of a dsDNA-nanoparticle treatment, (iii) the subject either had no response to a prior checkpoint inhibitor treatment in the absence of a dsDNA-nanoparticle or a decreasing level of response to the prior treatment and/or (iv) the cancer is stratified I3 or I4 based on quantification of CD3+ and CD8+ lymphocyte populations. In certain embodiments, the checkpoint inhibitor and nanoparticle comprising dsDNA are administered at the same time; are administered within about 15 minutes, within about 30 minutes, within about 60 minutes, within about 2 hours, within about 4 hours, within about 6 hours, within about 12 hours, within about a day, within about 2 days, within about 3 days, within about 4 days, within about 5 days, within about a about week or within about 2 weeks.

Cancer resistance to checkpoint inhibitor treatment can be evaluated based on the cancer type, analysis of the cancer and/or initial treatment with a checkpoint inhibitor. For example, as illustrated in the Examples below, a checkpoint inhibitor (exemplified by an anti-PD-L1 antibody) had no effect on tumor growth using a melanoma animal model, while a synergistic effect was seen with the dsDNA-nanoparticle (exemplified by an dsDNA-LNP) and checkpoint inhibitor (exemplified by an anti-PD-L1 antibody).

Reference to the cancer “resistant to checkpoint inhibitor treatment” refers to resistance to one or more checkpoint inhibitors, which may be a different checkpoint inhibitor than being administered by the method. The ability to be resistant to one or more checkpoint inhibitors provides an increased likelihood of resistance to treatment in general to a checkpoint inhibitor in the absence of dsDNA-nanoparticles. In certain embodiments, resistance is to the same checkpoint inhibitor being administered in combination with dsDNA-nanoparticle. In certain embodiments the checkpoint inhibitor is as provided in Table 2 and/or an additional therapeutic is added (e.g., as provided in Table 1).

Reference to the subject “has previously undergone a treatment” with a checkpoint inhibitor in the absence of a dsDNA-nanoparticle treatment” refers to prior treatment involving one or more checkpoint inhibitor, which may be a different checkpoint inhibitor than being administered by the method. Modifying treatment to include a dsDNA-nanoparticle in combination with a checkpoint inhibitor may be useful, for example, where the initial treatment with checkpoint inhibitor is not effective, not as effective as expected, and/or is decreasing in effectiveness. In certain embodiments, the method employs the same checkpoint inhibitor as is subsequently being administered in combination with dsDNA-nanoparticle. In certain embodiments, prior treatment with a checkpoint inhibitor in the absence of a dsDNA-nanoparticle treatment is for up to 4 years, up to 3 years, up to 2 years up to 1 years, up to 6 months, up to 5 months, up to 4 months, up to 3 months, up to 2 months or up to one month. In certain embodiment the checkpoint inhibitor is as provided in Table 2 and/or an additional therapeutic is added (e.g., as provided in Table 1).

### III. Pharmaceutical Compositions

Appropriate pharmaceutical compositions can be selected based on the compound being administered and administration route. The pharmaceutical composition contains one or more active component along with a pharmaceutical acceptable carrier. Reference to “pharmaceutically” or “pharmaceutically acceptable” refers to non-toxic molecular entities suitable for administration and/or storage. Pharmaceutical compositions can comprise more than one therapeutically active agent.

Examples of pharmaceutically acceptable carriers include a non-toxic (in the amount used) solid, semi-solid or liquid filler, diluent, encapsulating material, or formulation. Guidance concerning formulations for small molecule, vaccines, proteins and antibodies can be found, for example, in Remington (2020) The Science and Practice of Pharmacy 23rd Edition; D'Amico et al., (2021) Drug Deliv. and Transl. Res. 11, 353-372; and Strickley and Lambert (2021) Journal of Pharmaceutical Sciences 110:2590-2608.

The form of the pharmaceutical compositions, the route of administration, the dosage and the regimen depend upon the condition to be treated, such as the severity of the illness, the age, weight, and sex of the patient. Pharmaceutical compositions can be formulated for different modes of administration such as for topical, oral, intranasal, parenteral, intraocular, intravenous, intramuscular, or subcutaneous administration.

In an embodiment, the pharmaceutical composition contains a formulation capable of injection into a subject. Examples of injectable formulation components isotonic, sterile, saline solutions (e.g., monosodium or disodium phosphate, sodium, potassium, calcium or magnesium chloride and mixtures of such salts), buffered saline, sugars (e.g., dextrose), and water for injection. Pharmaceutical compositions include dry, for example, freeze-dried compositions which upon addition, depending on the case, of sterilized water or physiological saline, permit the constitution of injectable solutions. The doses used for the administration can be adapted as a function of various parameters such as mode of administration, relevant pathology, and duration of treatment.

Other pharmaceutically acceptable forms include tablets or other solids for oral administration, including time-release capsules.

### IV. Administration and Treatment

Administration routes and treatment regimens can be selected based upon the chosen compound, pharmaceutical composition and indication being treated. Administration routes include topical, oral, intranasal, parenteral, intraocular, intravenous, intramuscular, and subcutaneous administration. Guidance concerning formulations and administration for small molecules, vaccines, proteins and antibodies can be found, for example, in Remington (2020) The Science and Practice of Pharmacy 23rd Edition; D'Amico et al., (2021) Drug Deliv. and Transl. Res. 11, 353-372; and Strickley and Lambert (2021) Journal of Pharmaceutical Sciences 110:2590-2608). Additional guidance can be found, for example, in product inserts for approved therapeutics (e.g., see Tables 1 and 2 supra.)

Preferred doses provide an effective amount to achieve a detectable effect. Generally, small molecules will be administered in a dose of between 0.0001 and 10 mg/kg, or 0.001 to 1 mg/kg body weight. Generally large compounds such as antibodies and polypeptides may vary from about 10 ng/kg up to about 100 mg/kg of body weight, or about 1 mg/kg/day to 10 mg/kg/day.

An effective dose for dsDNA is sufficient to provide a detectable effect in the host immune system and should enhance vaccination or treatment. Generally, dsDNA will be administered in a range of 0.0001 mg/kg to 2 mg/kg. In certain embodiments the dsDNA will be administered in a range of 0.0001 to 0.001 mg/kg, 0.001 to 0.01 mg/kg, 0.01 to 0.1 mg/kg, or 0.1 to 2 mg/kg.

Reference to “treatment” or “treat” refers to both prophylactic, and therapeutic treatment of a patient having a disease or disorder. Prophylactic treatment provides a decreased likelihood of contracting a disease or disorder or decreasing the potential severity of a disease or disorder. Therapeutic treatment provides a clinical meaningful amelioration in at least one symptom or cause associated with a disease or disorder.

Methods treating cancer are able to reduce the spread of the cancer, reduce the number of cancer cells, decrease pathology, reduce recurrence rate, and/or inhibit cancer growth.

The terms “ameliorate”, and “amelioration” refer to a detectable or measurable improvement in a disease or disorder symptom or an underlying cellular response. A detectable or measurable improvement includes a subjective or objective decrease, reduction, inhibition, suppression, limit or control in the occurrence, frequency, severity, progression, or duration of the disease or disorder, or complication caused by or associated with the disease or disorder, or an improvement in a symptom or an underlying cause or a consequence of the disease or disorder, or a reversal of the disease or disorder.

The terms “effective amount” and “sufficient amount” is that amount required to obtain a desired effect. A therapeutically effective amount can be provided in single or multiple doses to achieve a therapeutic or prophylactic effect.

An effective amount can be administered alone or in combination with another therapeutic agent, compound, composition, treatment, protocol, or therapeutic regimen. The amount can be proportionally increased, for example, based on the need of the subject, type, status and severity of the disease or disorder treated or side effects.

Administration of one or more therapeutic agents, and a nanoparticle comprising dsDNA, can be together or separately. In certain embodiments, a therapeutic agent and a nanoparticle comprising dsDNA are administered at the same time; are administered within about 15 minutes, within about 30 minutes, within about 60 minutes, within about 2 hours, within about 4 hours, within about 6 hours, within about 12 hours, within about a day, within about 2 days, within about 3 days, within about 4 days, within about 5 days, within about a week or within about 2 weeks. In certain embodiments, one or more administered therapeutic agent is selected from the therapeutic agents in Section I. supra; is a cancer vaccine; is a checkpoint inhibitor; and/or is selected from Table 1 or Table 2.

In some cases, where administration is at the same time a composition can comprise both (i) a nanoparticle comprising DNA and (ii) one or more therapeutic agents; or the (i) a nanoparticle comprising DNA and (ii) one or more therapeutic agents, may be provided as separate compositions.

### V. Kits

Further provided herein is a kit providing in separate containers at least: (a) an effective amount of nanoparticle comprising dsDNA; and (b) an effective amount an anti-cancer therapeutic agent or a vaccine. Kit components are further described, for example, in Sections I-V supra. The kit may also provide a label with instructions for administration according to the methods described herein.

### VI. Additional Aspects and Embodiments

Additional aspects and embodiments include:

A first aspect describes a method of treating a cancer in a subject, comprising administering to the subject (a) a nanoparticle comprising dsDNA and (b) a cancer vaccine or a cancer therapeutic agent. Preferably, the dsDNA comprises a dsDNA region at least 45 base pairs in length.

Embodiment 1 further describes the first aspect, wherein the cancer is as provided in the first, second, third, or fourth groups of cancer in Section II. supra; or the cancer is selected from the group consisting of bladder cancer, breast cancer, colon and rectal cancer, endometrial cancer, kidney cancer, leukemia, liver cancer, lung cancer, melanoma, non-Hodgkin lymphoma, pancreatic cancer, prostate cancer and thyroid cancer. In further embodiments, the cancer is acute myeloma leukemia; the cancer is liver cancer; the cancer is melanoma; or the cancer is lung cancer.

Embodiment 2 further describes the first aspect and Embodiment 1, wherein the cancer is a tumor.

A second aspect describes a method of treating lung cancer, melanoma or liver cancer in a subject comprising administering to the subject a nanoparticle comprising dsDNA. Preferably, the dsDNA comprises a dsDNA region at least 45 base pairs in length.

Embodiment 3 further describes the first and second aspects and Embodiments 1 and 2, wherein the method comprises administering a cancer vaccine to the subject.

Reference to a particular embodiment includes reference to further embodiments provided therein. For example, reference in the second embodiment to the first embodiment provides a reference to all the embodiments provided in the first embodiment including the further embodiments provided therein.

Embodiment 4 further describes the first and second aspects and any of Embodiments 1-3, wherein the method comprises administering a cancer therapeutic agent. More than one type of therapeutic agents can be administered.

Embodiment 5 further describes the first and second aspects and any of Embodiments 1-4, wherein the therapeutic agent is selected from the group of therapeutic agents listed in Table 1 and/or is a checkpoint inhibitor.

Embodiment 6 further describes the first and second aspects and any of Embodiments 1-5, wherein the therapeutic agent is a checkpoint inhibitor selected from atezolizumab, avelumab, cemiplimab, dostarlimab, durvalumab, nivolumab, ipilimumab, and pembrolizumab; and/or the checkpoint inhibitor is an anti-PD-L1 antibody or an anti-PD-1 antibody.

Embodiment 7 further describes the first and second aspects and any of Embodiments 1-6 wherein at least two different therapeutic agents are administered. In further embodiments, one of therapeutic agents is a checkpoint inhibitor and one of the therapeutic agents is selected from the therapeutic agents listed in Table 2.

Embodiment 7 further describes the first and second aspects and any of Embodiments 1-6, wherein the vaccine and/or therapeutic agent are administered at, or about, the same time as the nanoparticle comprising the dsDNA.

A third aspect, describes a method of treating a cancer in a subject comprising administering to said subject:
a) a nanoparticle comprising a double-stranded DNA (dsDNA), wherein the dsDNA comprises a double-stranded region of at least 45 base pairs in length; and
b) a checkpoint inhibitor;
wherein (i) the cancer is resistant to checkpoint inhibitor treatment, (ii) the subject has previously undergone a treatment with a checkpoint inhibitor in the absence of a dsDNA-nanoparticle treatment, (iii) the subject either had no response to a prior checkpoint inhibitor treatment in the absence of a dsDNA-nanoparticle or a decreasing level of response to the prior treatment and/or (iv) said cancer is stratified 13 or 14 based on quantification of CD3+ and CD8+ lymphocyte populations.

Embodiment 8 further describes the third aspect, wherein the cancer is melanoma. In a further embodiment a Table 1 compound for treating melanoma is also administered.

Embodiment 9 further describes the third aspect and Embodiment 8, wherein the cancer is resistant to checkpoint inhibitor treatment in the subject. In a further embodiment, the cancer is resistant to a PD-L1 inhibitor or a PD-1 inhibitor.

Embodiment 10 further describes the third aspect and Embodiments 8 and 9, wherein the checkpoint inhibitor being administered is a PD-L1 inhibitor or a PD-1 inhibitor.

Embodiment 11 further describes the third aspect and Embodiments 8 and 9, wherein the checkpoint inhibitor being administered is selected from the group consisting of: atezolizumab, avelumab, cemiplimab, dostarlimab, durvalumab, nivolumab, ipilimumab, and pembrolizumab.

Embodiment 12, further describes the third aspect, wherein the subject either had no response to the prior treatment or a decreasing level of response to the prior treatment. In a further embodiment, the subject has previously undergone prior treatment with a PD-L1 inhibitor or a PD-1 inhibitor.

Embodiment 13, further describe the third aspect, wherein the subject either had no response to the prior treatment or a decreasing level of response to the prior treatment. In a further embodiment, the subject has previously undergone prior treatment with a PD-L1 inhibitor or a PD-1 inhibitor.

Embodiment 14, further describes embodiments 12 and 13, wherein the checkpoint inhibitor being administered is selected from the group consisting of: atezolizumab, avelumab, cemiplimab, dostarlimab, durvalumab, nivolumab, ipilimumab, and pembrolizumab.

Embodiment 15 further describes the third aspect and any of Embodiments 8-13 wherein at least two different therapeutic agents are administered. In further embodiments, one of therapeutic agents is a checkpoint inhibitor and one of the therapeutic agents is selected from the therapeutic agents listed in Table 2.

Embodiment 16 further describes the first, second and third aspects and any of Embodiments 1-15, wherein the dsDNA comprises a dsDNA region at least 50 base pairs in length. In further embodiments: (1) the dsDNA region is at least 100 base pairs in length, at least 200 base pairs in length, at least 250 base pairs in length, at least 300 base pairs in length, at least 400 base pairs in length, at least 500 base pairs in length, at least 600 base pairs in length, at least 700 base pairs in length, at least 800 base pairs in length, at least 900 base pairs in length, at least 1000 base pairs in length, at least 1100 base pairs in length; at least 1200 base pairs in length, at least 1300 base pairs in length, at least 1400 base pairs in length, or at least 15,000 base pairs in length; and/or has a size range between any two of the mentioned sizes in (1).

Embodiment 17 further describes the first, second and third aspects and any of Embodiments 1-16, wherein the dsDNA contains 6 or fewer CpGs, 5 or fewer CpGs, 4 or fewer CpGs, 3 or fewer CpGs, 2 CpGs, 1 or fewer CpG, or zero CpG.

Embodiment 18 further describes the first, second and third aspects and any of Embodiments 1-17, wherein the dsDNA region and other nucleotides, if present, are naturally occurring and/or modified. In further embodiments, the dsDNA is modified and stimulates an innate immune response of at least 50%, at least 65%, at least 75%, at least 85%, at least 90%, or least 100% compared to the corresponding unmodified dsDNA as measured by IFN-β, IL-6 and/or IL-1ß as provided in the Examples below.

Embodiment 19 further describes the first, second and third aspects and any of Embodiments 1-18, wherein the nucleotides making up the dsDNA region and other nucleotides, if present, are unmodified or contain one or more modified nucleotides selected from the group consisting 2′-methoxyethyl (2′-MOE), 2′-fluor (2′-F), locked nucleic acid (LNA), constrained ethyl (cEt), tricyclo-DNA (tc-DNA), C7-modified deaza-adenine (methyl, Cl or F), C7-modified deaza-guanosine (methyl, Cl or F), C5-modified cytosine (methyl, F or Cl), and C5-modified uridine (methyl, F or Cl), and/or backbone modifications (phosphorothioate (Rp and/or Rs), thio-phosphoramidate, phosphorodiamidate morpholino oligos (PMO), and peptide-nucleic acid (PNA). In further embodiments, the one or more modifications are phosphorothioate (Rp and/or Rs). In further embodiment, the dsDNA is modified and stimulates an innate immune response of at least 50%, at least 65%, at least 75%, at least 85%, at least 90%, or least 100% compared to the corresponding unmodified dsDNA as measured by IFN-β, IL-6, and/or IL-1B as provided in the Examples below.

Embodiment 19 further describes Embodiments 17 and 18, wherein no more than 95%, no more than 85%, no more than 75%, no more than 65%, no more than 55%, no more than 45%, no more than 35%, no more than 30%, no more than 25%, no more than 20%, no more than 15%, no more than 10%, no more than 5%, or 0% of the nucleotides are modified.

Embodiment 20 further describes Embodiments 17-19, wherein the dsDNA and/or dsDNA region (1) is at least 50 base pairs in length, at least 100 base pairs in length, at least 200 base pairs in length, at least 250 base pairs in length, at least 300 base pairs in length, at least 400 base pairs in length, at least 500 base pairs in length, at least 600 base pairs in length, at least 700 base pairs in length, at least 800 base pairs in length, at least 900 base pairs in length, at least 1000 base pairs in length, at least 1100 base pairs in length, at least 1200 base pairs in length, at least 1300 base pairs in length, at least 1400 base pairs in length, or at least 15,000 base pairs in length; and/or has a size range between any two of the mentioned sizes in (1); wherein no more than 95%, no more than 85%, no more than 75%, no more than 65%, no more than 55%, no more than 45%, no more than 35%, no more than 30%, no more than 25%, no more than 20%, no more than 15%, no more than 10%, no more 5%, or 0% of the dsDNA region is modified; where the remaining nucleotides, if present, may have the same percentage of modifications or different percentage of modifications as the dsDNA region in (1). In further embodiments no more than 95%, no more than 85%, no more than 75%, no more than 65%, no more than 55%, no more than 45%, no more than 35%, no more than 30%, no more than 25%, no more than 20%, no more than 15%, no more than 10%, no more 5% or 0% of the nucleotides outside of the dsDNA region in (1), if present, are modified.

Embodiment 21 further describes the first, second and third aspects and any of Embodiments 1-20, wherein the dsDNA region is formed by two separate polynucleotides or two regions of a single polynucleotide.

Embodiment 22 further describes the first, second and third aspects and any of Embodiments 1-21, wherein the dsDNA is linear or circular. In further embodiments, the dsDNA is selected from the group consisting of a minicircle, a plasmid, an open linear duplex DNA, and a closed-ended linear duplex DNA.

Embodiment 23 further describes the first and second aspects and any of Embodiments 1-22, wherein the dsDNA is noncoding, lacks a promoter coupled to a coding region for expression in the subject being treated (e.g., mammalian or human cell) and/or is not a DNA vector comprising a transgene. Reference to “noncoding” indicates the dsDNA does not code for a gene (express a gene product) in the subject.

Embodiment 24 further describes the first, second and third aspects and any of Embodiments 1-23, wherein the nanoparticle is a lipid nanoparticle, polymeric nanoparticle, lipid polymer nanoparticles (LPNP), protein and peptide-based nanoparticles, DNA dendrimers or DNA-based nanocarriers, carbon nanotubes, microparticles, microcapsules, inorganic nanoparticle, or peptide cage nanoparticles; the nanoparticle is an LNP or LPNP; or the nanoparticle is an LNP, and the LNP in mole %, comprises, consists essentially, or consists of the following components (1) cKK-E12, about 35%; C14-PEG2000, about 2.5%; cholesterol, about 46.5%; and DOPE, about 16%; (2) Lipid 9, about 50%; C14-PEG2000, about 1.5%; cholesterol, about 38.5%; and DSPC, about 10%; or (3) bCKK-E12, about 35%; C14-PEG2000, about 2.5%; cholesterol, about 46.5%; and dioleoylphosphatidylethanolamine (DOPE), about 16%.

Embodiment 25 further describes the first, second and third aspects and any of Embodiments 1-23, wherein the nanoparticle comprises mol % of the following components: one or more cationic lipids from about 20% to about 65%, one or more phospholipids from about 1% to about 50%, one or more PEG-conjugated lipids from about 0.1% to about 10%, and cholesterol from about 0% to about 70%; or one or more cationic lipids from about 20% to about 50%, one or more phospholipids from about 5% to about 20%, one or more PEG-conjugated lipids from about 0.1% to about 5%, and cholesterol from about 20% to about 60%; in additional embodiments the phospholipid lipid is a neutral lipid; and the phospholipid lipid is DOPE or DSPC.

Embodiment 26 further describes the first, second and third aspects and any of Embodiments 1-25, wherein the method induces a T cell response. In a further embodiment, the T cell response is a Th1 or Th2 response.

Embodiment 27 further describes the first, second and third aspects and any of Embodiments 1-25, wherein the subject is a human subject.

A fourth aspect describes a nanoparticle comprising dsDNA for use in the method of the first, second and third aspects and any of Embodiments 1-27. Preferably, wherein the dsDNA comprises a double-stranded region of at least 45 base pairs in length.

A fifth aspect describes the use of a nanoparticle comprising dsDNA for the preparation of a medicament. In different embodiments, the dsDNA comprises a double-stranded region of at least 45 base pairs in length; and/or the medicament is for use in the methods of the first and second aspects and any of Embodiments 1-27.

### EXAMPLES

Examples are provided below further illustrating different features of the present invention and methodology for practicing the invention. The provided examples do not limit the claimed invention.

### Example 1: Antitumor Effects in a Melanoma Cancer Model

The ability of dsDNA nanoparticle delivery to inhibit melanoma was evaluated in mice implanted subcutaneously (s.c.) with 1×10⁶ B16-F10 cells according to Table 3.

TABLE 3

| Group | | day −6 | | | | | day 0 | | day | day |
|---|---|---|---|---|---|---|---|---|---|---|
| Number | Mice | Implantation | Treatment | Dose | | n | 0 h | +4 h | 3 | 6 |
| 1 | C57BL/6J | B16F10 | 20% | — | | 7 | i.t. | Bleed | i.t. | i.t. |
| | (Female) | (s.c.) | Glucose/PBS | | | | | | | |
| 2 | | | noncoding | 1 | μg | 7 | i.t. | Bleed | i.t. | i.t. |
| 3 | | | DNA-LNP | 10 | μg | 7 | i.t. | Bleed | i.t. | i.t. |
| 4 | | | | 10 | μg | 7 | i.t. | Bleed | — | — |
| 5 | | | | 10 | μg | 7 | i.v. | Bleed | i.v. | i.v. |
| 6 | | | ADU-S100 | 10 | μg | 7 | i.t. | Bleed | i.t. | i.t. |

Treatment was provided by 100 μL intratumoral (i.t.) or intravenous (i.v.) administration, given one to three times. “DNA-LNP 1 μg×3” indicates 1 μg DNA-LNP were injected intratumorally three times (day 0, 3, 6). The DNA was provided by a CpG-free plasmid that does not provide for gene expression in mammalian cells. The plasmid was made up of approximately 5.1 kb dsDNA. ADU-S100 is a STING agonist (InvivoGen). The LNP composition (in mole %) was: Lipid 9, 50% (Lipid 9 is further described in Section I.A. supra and Sabnis et al., (2018) Molecular Therapy 26:6, 1509-1519); C14-PEG2000, 1.5%; cholesterol, 38.5%; and distearoylphosphatidylcholine (DSPC), 10%.

Mice (n=7) were observed every three days and sacrificed when the largest tumor reached 3000 mm³. Two mice in the DNA-LNP 10 μg×1 were moribund and sacrificed on days 8 and 11, before there was significant tumor growth. These two mice were not included in the data.

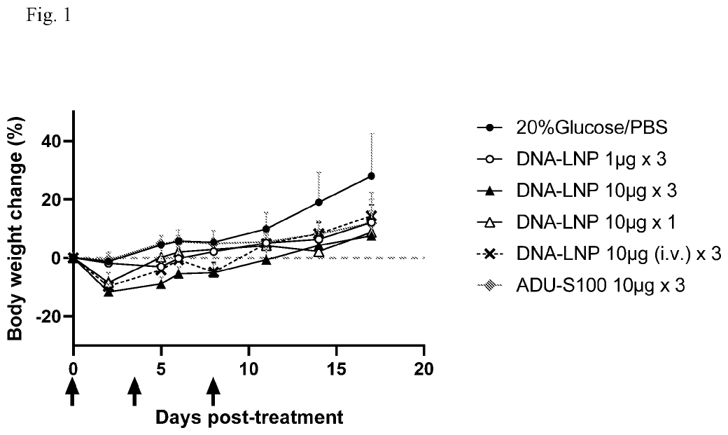

FIG. 1 illustrates the change in body weight of mice implanted with B16-F10 (melanoma model) cells and treated with DNA-LNP or ADU-S100.

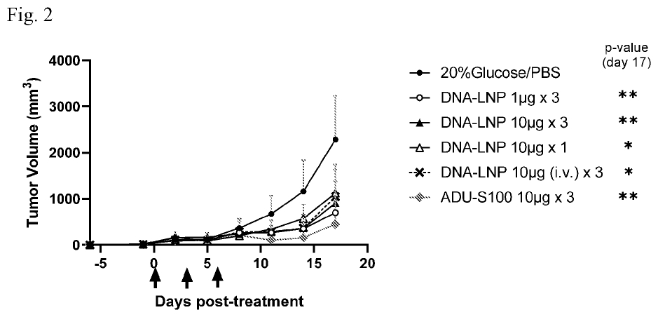

FIG. 2 illustrates tumor growth in mice implanted with B16-F10 and treated with DNA-LNP or ADU-S100. Statistical differences are indicated by * (P-value<0.05), ** (P-value<0.01). DNA-LNP exerted a local effect against subcutaneously implanted B16-F10 tumor and showed a systemic effect on B16-F10.

### Example 2: Antitumor Effects in a Lung Cancer Model

The ability of nanoparticle dsDNA delivery to inhibit lung cancer was evaluated in mice implanted intravenously with 1×10⁵ cells of B16-F10 according to according to Table 4.

TABLE 4

| Group | | day −5 | | | | | day 0 | | day | day |
|---|---|---|---|---|---|---|---|---|---|---|
| Number | Mice | Implantation | Treatment | dose | | n | 0 h | +4 h | 3 | 6 |
| 1 | C57BL/6J | B16F10 | 20% Glucose/PBS | — | | 8 | i.v. | Bleed | i.v. | i.v. |
| 2 | (Female) | (i.v.) | noncoding DNA- | 1 | μg | 8 | i.v. | Bleed | i.v. | i.v. |
| 3 | | | LNP | 10 | μg | 8 | i.v. | Bleed | i.v. | i.v. |
| 4 | | | | | | 8 | i.v. | Bleed | — | — |
| 5 | | | ADU-S100 | 50 | μg | 8 | i.v. | Bleed | i.v. | i.v. |

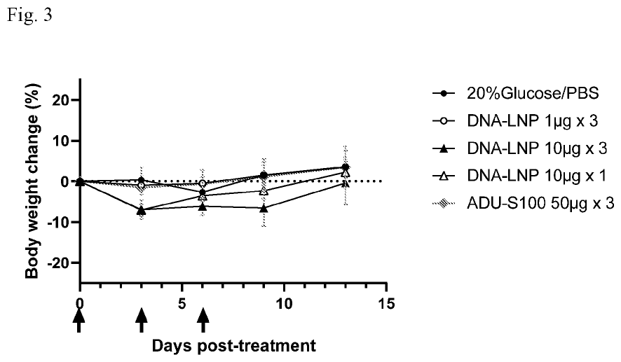

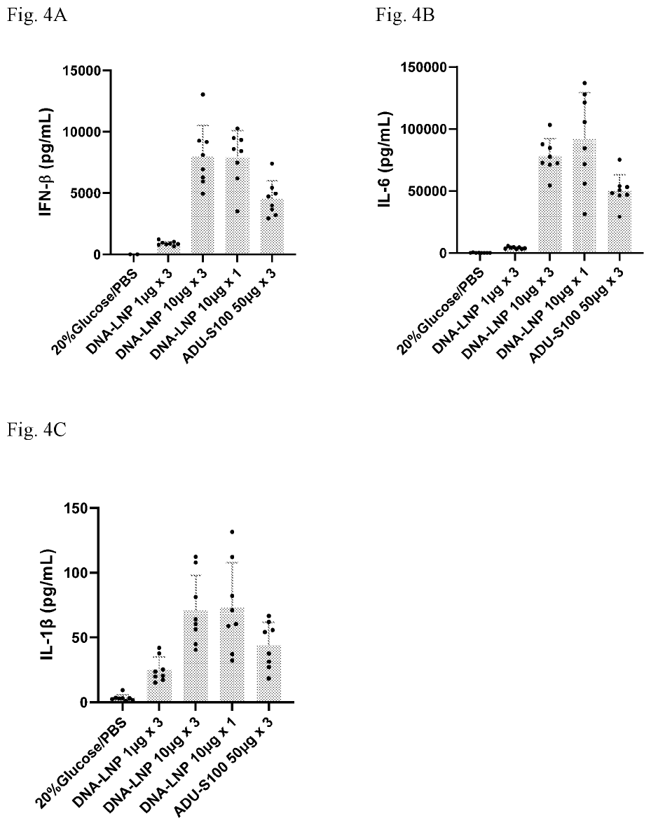

Administration was provided by 100 μL intravenous administration of ADU-S100 or DNA-LNP, given one to three times. “DNA-LNP 1 μg×3” indicates 1 μg DNA-LNP were injected intravenously three times (day 0, 3, 6). The DNA-LNP was as described in Example 1. Each group started with 8 mice. Mice were sacrificed on day 13, 18 days after implantation. FIG. 3 illustrates change in body weight of mice implanted with B16-F10 (lung metastasis model) and treated with DNA-LNP.FIG. 4A, FIG. 4B. and FIG. 4C, illustrate serum cytokines in mice implanted with B16-F10 and treated with DNA-LNP or ADU-S100. Serum was collected 4 hours after the first dose. FIG. 4A shows IFN-β, FIG. 4B shows IL-6, and FIG. 4C shows IL-1B.

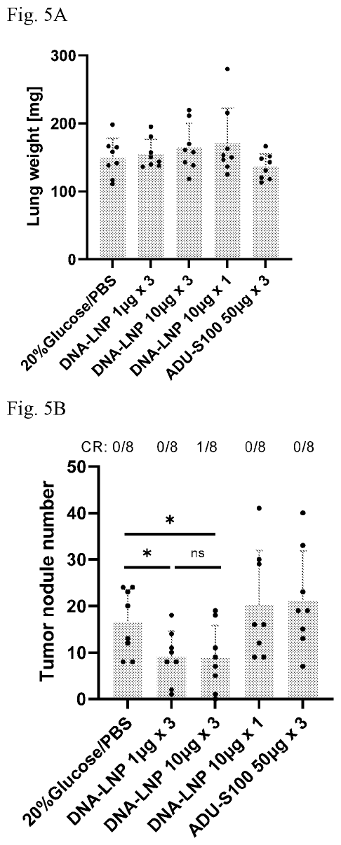

FIG. 5A and FIG. 5B illustrate lung weight and tumor nodule number in mice implanted with B16-F10 and treated with DNA-LNP or ADU-S100. Lung weight and tumor nodule number were measured 18 days after tumor injection, which corresponded to 13 days after treatment initiated.

### Example 3: Antitumor Effects in a Liver Cancer Model

The ability of nanoparticle dsDNA delivery to inhibit liver cancer was evaluated in a mouse model for hepatocellular carcinoma (HCC). HCC was induced by administering oncogene vector groups via hydrodynamic injection (HDI). Oncogene vector groups contained a plasmid expressing the transgene of the protooncogene cMET and a plasmid expressing the transgene for CTNNB1. (See, Subleski et al., (2015), J Hepatol, 63 (5): 1181-1189; Tward et al., (2007), PNAS 104 (37): 14771-14776.)

C57BL/6 mice (4 to 7 mice/group) were injected with either pT3 control or oncogene vector groups at day-22 and treatment was started at day 0. The pT3 control contained 4 μg pT3-Glu, 40 μg pT3-empty, and 4.4 μg HSB2. The oncogene vector groups contained 4 μg pT3-Glu, 40 μg pT3-empty, 4.4 μg HSB2, 20 μg pT3-N90-βcat (Addgene #31785), and 20 μg pT3-EF1a-cMET (Addgene #31784). Treatment was carried out with DNA-LNP low dose (1 μg), DNA-LNP high dose (10 μg) or control. pT3-empty is an empty vector plasmid control. HSB2 is the plasmid encoding Sleeping Beauty Transposase. pT3-N90-βcat is the plasmid encoding CTNNB1 oncogene. pT3-EF1a-cMET is the plasmid encoding cMET oncogene. pT3-Glu is the plasmid encoding Gaussia luciferase reporter that serves as a proxy for tumor growth. DNA-LNP is as described in Example 1.

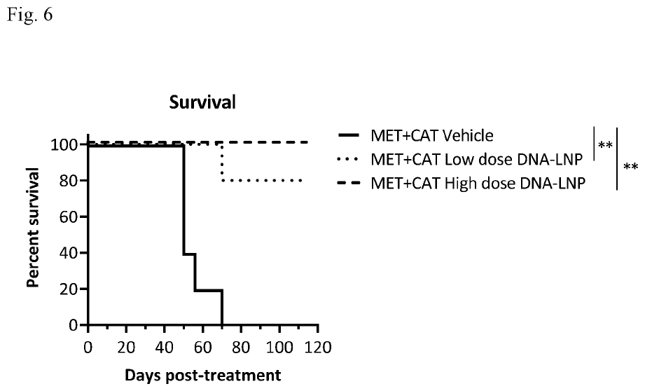

FIG. 6 illustrates increased survival in the liver model due to DNA-LNP administration. Mean survival of non-treated mice was 10 weeks post HDI. ** indicates P-value<0.01 by a log-rank Mantel-Cox test.

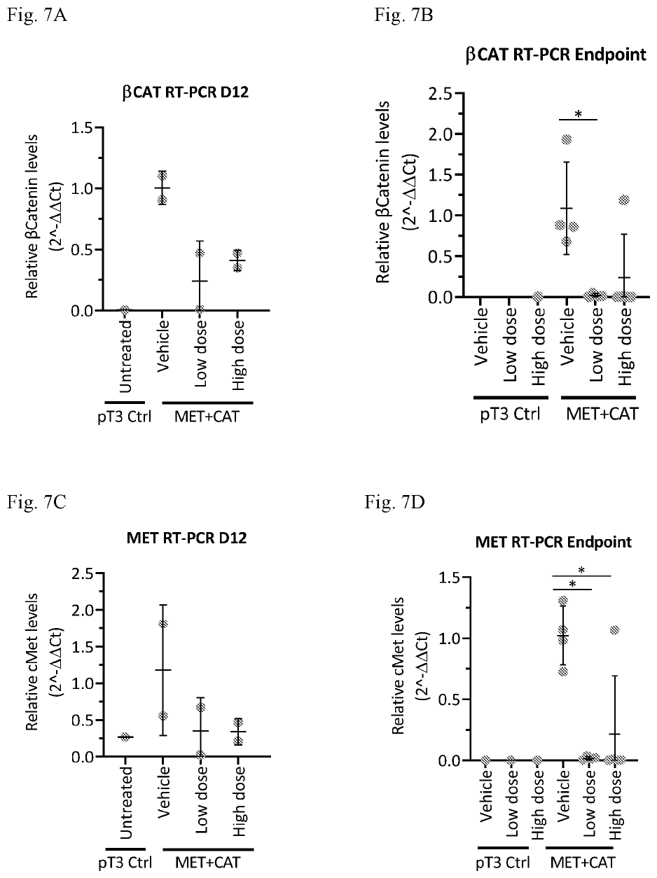

FIGS. 7A, 7B, 7C, and 7D, illustrate the ability of DNA-LNP to induce antitumor efficacy as measured by oncogene end point expression. DNA-LNP was as described in Example 1. FIG. 7A shows relative levels in β-catenin mRNA measured by RT-PCR at day 12 (D12). FIG. 7B shows relative levels in β-catenin mRNA measured by qRT-PCR at the endpoint. FIG. 7C shows relative levels in cMET mRNA measured by RT-PCR at day 12 (D12). FIG. 7D shows relative levels in cMET mRNA measured by RT-PCR at the endpoint.

### Example 4: Antitumor Effects in a Bilateral Melanoma Cancer Model

The ability of dsDNA nanoparticle delivery to inhibit melanoma was evaluated in mice implanted subcutaneously with 2×10⁵ B16-F10 cells on one flank (local flank) and 1×10⁵ B16-F10 cells on the other flank (distal flank) according to Table 5.

TABLE 5

| Group | | day −8 | | | | | day 0 | | | |
|---|---|---|---|---|---|---|---|---|---|---|
| Number | Mice | Implantation | Treatment | n | Dose | | 0 h | +4 h | day 3 | day 6 |
| 1 | C57BL/6J | B16F10 | PBS/Glucose | 8 | — | | i.t. | Bleed | i.t. | i.t. |
| 2 | (Female) | (s.c.) | Noncoding | 8 | 0.1 | μg | i.t. | Bleed | i.t. | i.t. |
| 3 | | 2 × 10⁵ (local) | plasmid- | 8 | 1 | μg | i.t. | Bleed | i.t. | i.t. |
| 4 | | 1 × 10⁵ (distal) | LNP | 8 | 10 | μg | i.t. | Bleed | i.t. | i.t. |
| 5 | | | mIL-18 | 8 | 0.1 | μg | i.t. | Bleed | i.t. | i.t. |
| 6 | | | plasmid- | 8 | 1 | μg | i.t. | Bleed | i.t. | i.t. |
| 7 | | | LNP | 8 | 10 | μg | i.t. | Bleed | i.t. | i.t. |
| 8 | | | ADU-S100 | 8 | 10 | μg | i.t. | Bleed | i.t. | i.t. |

Treatment was given three times on day 0, 3, and 6, beginning when the average tumor volume reached 93 mm³. Treatment was provided by 100 μL intratumoral administration of the indicated dose of DNA-LNP, to the local flank inoculated with 2×10⁵ B16-F10 cells. The noncoding plasmid is the DNA described in Example 1. The mIL-18 plasmid is a CpG-containing murine IL-18 cytokine (mIL-18) encoding and expressing plasmid. The noncoding plasmid and the mIL-18 plasmid contained approximately 5.1 kb and 1.5 kb dsDNA, respectively. ADU-S100 is a STING agonist (InvivoGen). The LNP composition (in mole %): Lipid 9, 50%; C14-PEG2000, 1.5%; cholesterol, 38.5%, and distearoylphosphatidylcholine (DSPC), 10%.

Mice (n=8) were observed every three days. Two mice in the noncoding plasmid-LNP 1 μg group and one mouse in the mIL-18 plasmid-LNP 1 μg group were found dead on day 4 before there was significant tumor growth. These mice were not included in the data.

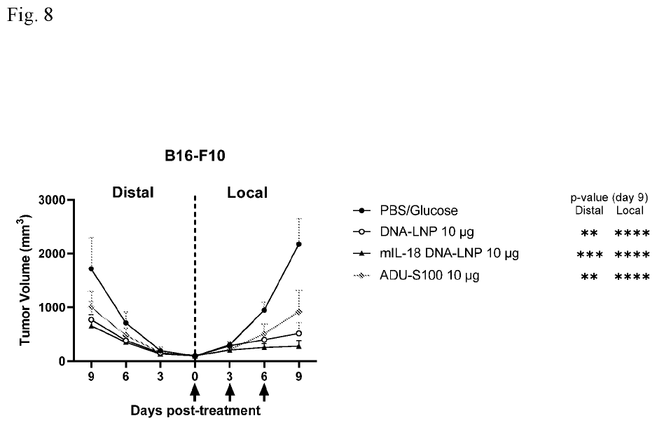

FIG. 8 shows tumor growth (by volume) in mice for the distal (flank where tumor did not receive a DNA-LNP injection) and local (flank where tumor was injected with 10 μg of a specified treatment) tumors. Statistical differences are indicated by ** (P-value<0.01), *** (P-value<0.001), and **** (P-value<0.0001). DNA-LNP, regardless of the DNA length or the presence of a transgene expression cassette, exerted antitumor effects against local and distal subcutaneous B16-F10 tumor. Lower doses of DNA-LNP (0.1 μg and 1 μg) did not show a statistical difference in protection, compared to control, when tumor size was evaluated up to nine days post-treatment.

### Example 5: Antitumor Effects in an Acute Myeloid Leukemia Model

The ability of nanoparticle dsDNA delivery to inhibit leukemia was evaluated in a mouse model for acute myeloid leukemia (AML). The AML model was established by intravenously (i.v.) injecting C57BL/6 mice with 1×10⁶ C1498 cells. (See Zhang et al., Blood 2009; 114 (8): 1545-1552 and Curran et al., 2016, Cell Reports 15, 2357-2366).

Three days post-tumor cell injection, treatment (vehicle, DNA-LNP (10 μg), ADU-S100 (50 μg)) was provided by 100 μL i.v. administration given three times (day 0, 3, 6). Vehicle was made of 20% Glucose/PBS solution. LNP composition (in mole %) was Lipid 9, ˜50% (Lipid 9 is further described in Section I.A. supra and Sabnis et al., (2018) Molecular Therapy 26:6, 1509-1519); C14-PEG2000, ˜1.5%; cholesterol, ˜38.5%; and distearoylphosphatidylcholine (DSPC), ˜10%. ADU-S100 is a STING agonist (MedChemExpress). The DNA was provided by an approximately 5.1 kb CpG-free plasmid that does not provide for gene expression in mammalian cells.

Serum was collected 4 hours after the first dose for cytokine/chemokine analyses. Mice (n=8) were observed daily for abnormalities and euthanized individually when the animal reached the humane endpoint.

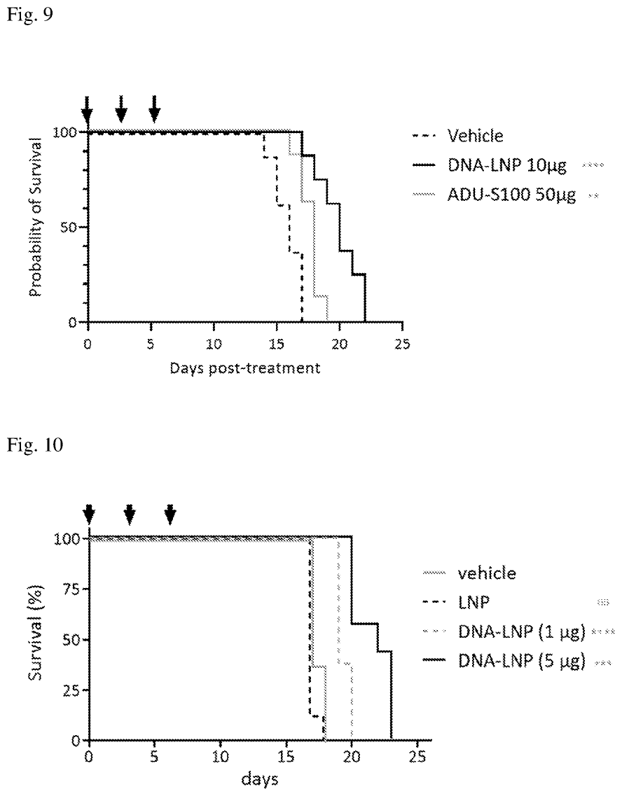

FIG. 9 shows increased survival in the AML model with administration of DNA-LNP or ADU-S100.

Table 6 provides data on serum cytokine levels 4 hours after dosing DNA-LNP or ADU-S100 in the AML model. In contrast to the STING agonist ADU-S100, DNA-LNP induced IL-18 and IFNγ. This indicates activation of the inflammasome pathway (IL-18) by DNA-LNP that may induce IFNγ production.

TABLE 6

| Serum level | vehicle | | DNA-LNP 10 μg | | ADU-S100 50 μg | |
|---|---|---|---|---|---|---|
| (pg/mL) | Average | SD | Average | SD | Average | SD |
| IFN alpha | 21 | 0 | 10835 | 981 | 1775 | 419 |
| IFN beta | 40 | 0 | 4685 | 2048 | 2819 | 475 |
| IFN gamma | 5 | 0 | 2703 | 1156 | 7 | 4 |
| IL-6 | 27 | 0 | 5680 | 2399 | 2617 | 440 |
| GRO alpha | 8 | 0 | 474 | 296 | 134 | 81 |
| MCP-1 | 60 | 0 | 6334 | 3422 | 5571 | 2086 |
| MIP-1 beta | 5 | 0 | 705 | 423 | 640 | 204 |
| TNF alpha | 14 | 0 | 433 | 219 | 732 | 262 |
| IL-18 | 150 | 0 | 6617 | 4451 | 150 | 0 |

### Example 6: Survival Effects in an AML Model

The ability of nanoparticle dsDNA delivery to inhibit leukemia was evaluated in the AML mouse model described in Example 5. Three days post-tumor cell injection, administration of vehicle, LNP (same amount of LNP as used in 5 μg DNA-LNP), DNA-LNP (1 μg), DNA-LNP (5 μg) was provided by 100 μL i.v. given three times (day 0, 3, 6). Mice (n=8) were observed daily for abnormalities and euthanized individually when the animal reached the humane endpoint. For CD8 depletion, 200 μg of anti-CD8a (BioXCell, In VivoMAb clone 2.43) was provided by 100 μL intraperitoneal (i.p.) administration given four times (day-2, 1, 4, 7). Vehicle, LNP, and DNA are as provided for in Example 5.

FIG. 10 illustrates the DNA dose-dependent increased survival in the AML model due to DNA-LNP administration. LNP indicates “empty” LNP (no DNA).

### Example 7: CD8-Mediated Effects in an AML Model

The DNA-LNP induced a CD8-mediated, dose-dependent anti-tumoral response in the AML model. One day after tumor cell injection, 200 μg of anti-CD8 was administrated intraperitoneally four times, three days apart (d-2, 1, 4, 7). Three days after tumor cell injection, DNA-LNP was administrated intravenously three times, three days apart (d0, 3, 6).

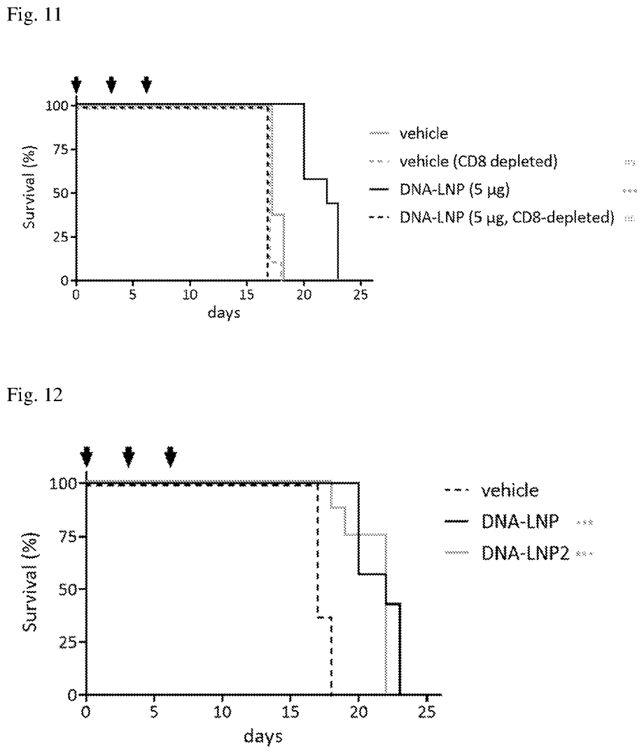

FIG. 11 illustrates the survival of the DNA-LNP-dosed AML model after depleting CD8+ cells. 200 μg of anti-CD8 was administrated intraperitoneally at d-2, 1, 4, and 7. DNA depletion led to decreased survival, indicating DNA-LNP induced a CD8-mediated anti-tumoral response in a mouse model of AML

### Example 8: Effect of Modified LNP or DNA in an AML Model

The ability of the nanoparticle dsDNA to induce antitumor effects in the AML model was tested using two different LNPs (LNP and LNP2) and two different DNA's (DNA and DNA2). The AML model was produced as described in Example 5. Vehicle, DNA and LNP were as provided for in Example 5. LNP2 is made up bCKK-E12, ˜35%; C14-PEG2000, ˜2.5%; cholesterol, ˜46.5%; and dioleoylphosphatidylethanolamine (DOPE), ˜16%. DNA2 is plasmid approximately 1.3 kb, containing 16 CpGs and does not provide for gene expression in mammalian cells

Three days post-tumor cell injection, treatment (vehicle, DNA-LNP (5 μg), DNA-LNP2 (5 μg)) was provided by 100 μL i.v. administration given three times (day 0, 3, 6). Serum was collected 4 hours after the first dose for cytokine/chemokine analyses. Mice (n=8) were observed daily for abnormalities and euthanized individually when the animal reached the humane endpoint.

FIG. 12 illustrates the increased survival in the AML model after 5 μg of DNA-LNP or DNA-LNP2 (different LNP) administration.

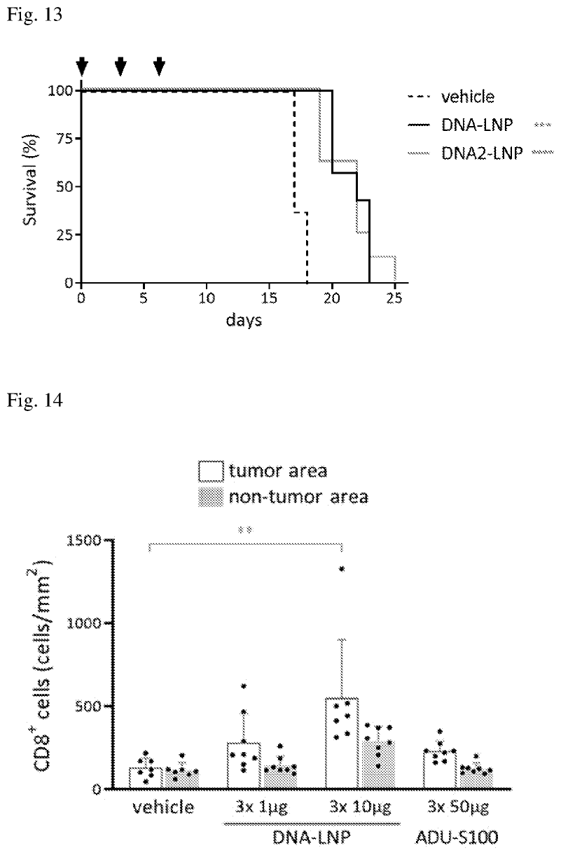

FIG. 13 illustrates the increased survival in the AML model after 5 μg of DNA-LNP or DNA2-LNP (different DNA) administration.

Table 7 provides data on serum cytokine levels 4 hours after dosing 5 μg of DNA-LNP, DNA-LNP2, or DNA2-LNP in the AML model.

TABLE 7

| Serum level | vehicle | | DNA-LNP | | DNA2-LNP | | DNA-LNP2 | |
|---|---|---|---|---|---|---|---|---|
| (pg/mL) | Average | SD | Average | SD | Average | SD | Average | SD |
| IFN alpha | 11 | 6 | 2314 | 442 | 1511 | 340 | 1912 | 272 |
| IFN beta | 9 | 0 | 543 | 115 | 238 | 68 | 444 | 82 |
| IFN gamma | 1 | 0 | 631 | 144 | 138 | 83 | 583 | 114 |
| IL-6 | 6 | 0 | 1509 | 408 | 709 | 344 | 1682 | 377 |
| GRO alpha | 9 | 4 | 552 | 232 | 111 | 43 | 175 | 43 |
| MCP-1 | 12 | 0 | 1173 | 334 | 897 | 313 | 782 | 158 |
| MIP-1 beta | 1 | 0 | 60 | 15 | 63 | 24 | 53 | 17 |
| TNF alpha | 3 | 0 | 102 | 31 | 56 | 15 | 75 | 17 |
| IL-18 | 30 | 0 | 363 | 75 | 150 | 61 | 467 | 170 |

### Example 9: CD8 Cell Infiltration in a Lung Model

Melanoma lung metastasis model was established by intravenously injecting C57BL/6 mice with 1×10⁵ B16-F10 cells. 5 days post-tumor cell injection, treatment was provided by 100 μL i.v. administration given three times (day 0, 3, 6). Mice (n=8) were sacrificed on day 13, and the lung was formalin fixed paraffin embedded (FFPE) for the immunohistochemistry (IHC) of CD8α.

FIG. 14 illustrates the number of CD8+ cells in the lung isolated from mice with B16-F10 (lung metastasis model) and treated with DNA-LNP or ADU-S100. Treatment was given intravenously three times, three days apart starting 5 days after tumor cell injection. Lung tissue was collected 18 days after tumor cell injection and FFPE for IHC of CD8. Number of CD8+ cells increased in the lung-metastasized B16-F10 tumor after DNA-LNP treatment.

### Example 10: Tumor Infiltrating Lymphocytes in a Flank Model

A flank melanoma model was established by subcutaneously (s.c.) injecting C57BL/6 mice with 2.5×10⁵ B16-F10 cells. Thirteen days post-tumor cell injection, treatment was provided by 50 μL intratumoral (i.t.) administration given two times, three days apart (day 0, 3). Mice (n=8) were sacrificed on day 7, and the tumor was collected for immune cell analysis using flow cytometry. Vehicle and DNA-LNP composition is as provided in Example 5. DNA-LNP was administered at a dose of 1 μg or 5 μg.

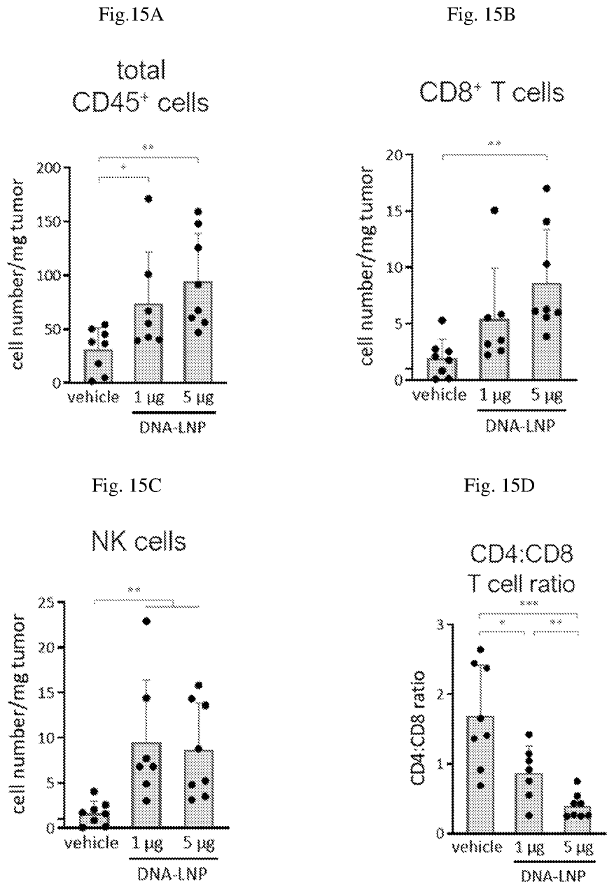

FIG. 15A, FIG. 15B, FIG. 15C, and FIG. 15D illustrate a dose-dependent increase of tumor infiltrating lymphocytes (TILs) due to DNA-LNP administration in the B16-F10 melanoma model as measured in CD45+ cells, CD8+ T cells, NK cells and a CD4:CD8 T cell ratio.

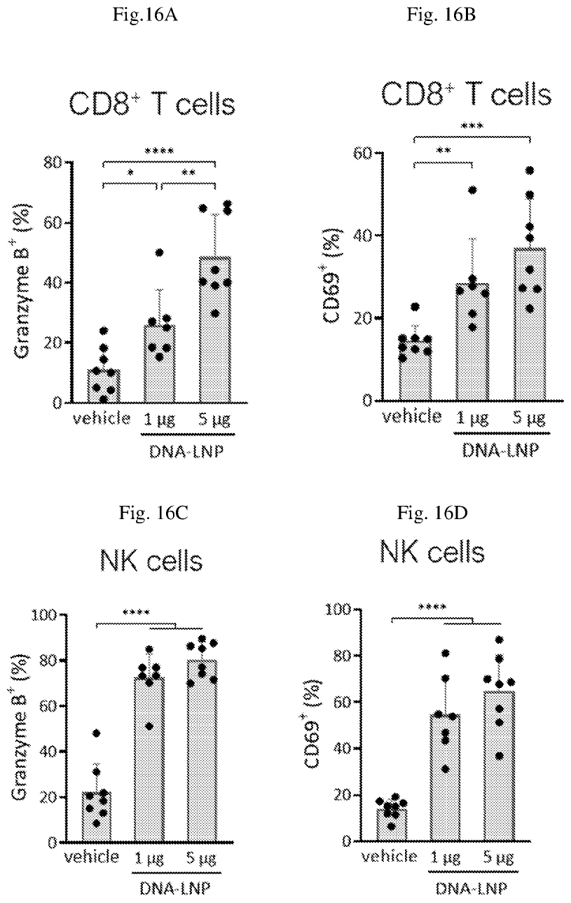

FIG. 16A, FIG. 16B, FIG. 16C, and FIG. 16D illustrates a dose-dependent increase of activation markers on CD8⁺ T cells and NK cells due to DNA-LNP administration in the B16-F10 flank melanoma model.

### Example 11: Immune Cell Depletion in a Melanoma Model

A flank melanoma model was established by subcutaneously (s.c.) injecting C57BL/6 mice with 2×10⁵ B16-F10 cells. Six days after tumor cell injection, 200 μg of antibody (IgG, anti-CD8, anti-NK1.1) were dosed intraperitoneally four times, three days apart (d-2, 1, 4, 7). Eight days after tumor cell injection, 10 μg of DNA-LNP or vehicle was administrated intratumorally three times, three days apart (d0, 3, 6). Mice were euthanized when the tumor size reaches 3000 mm³. DNA-LNP and vehicle is as described in Example 5.

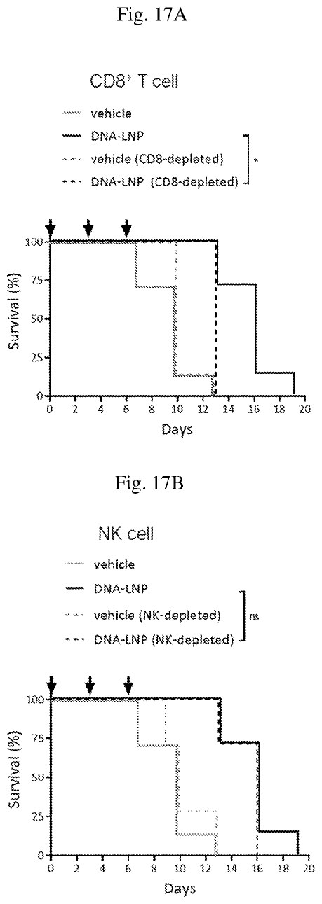

FIG. 17A and FIG. 17B illustrates the survival of the DNA-LNP-dosed B16-F10 melanoma model after depleting CD8+ cells or NK cells. Groups “vehicle” and “DNA-LNP” were treated with IgG isotype. DNA-LNP administration increased the survival of melanoma-bearing mice. Anti-tumoral effect was blunted in CD8-depleted mice.

### Example 12: Synergistic Effect with Anti-PD-L1 in a Melanoma Model

A flank melanoma model was established using B16-F10 tumor as described in Example 11. B16-F10 tumor is resistant to anti-PD-L1 treatment. Nine days after tumor cell injection, 250 μg of anti-PD-L1 (BioXCell, InVivoMAb clone 10F.9G2) was provided by 100 μL i.p. administration given three times (day 0, 3, 6). Vehicle and DNA-LNP (1 μg or 5 μg), was described in Example 5, and also administered i.t. day 0, 3, 6. Mice were euthanized when the tumor size reaches 3000 mm³.

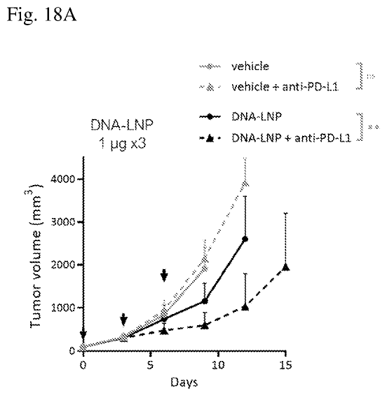

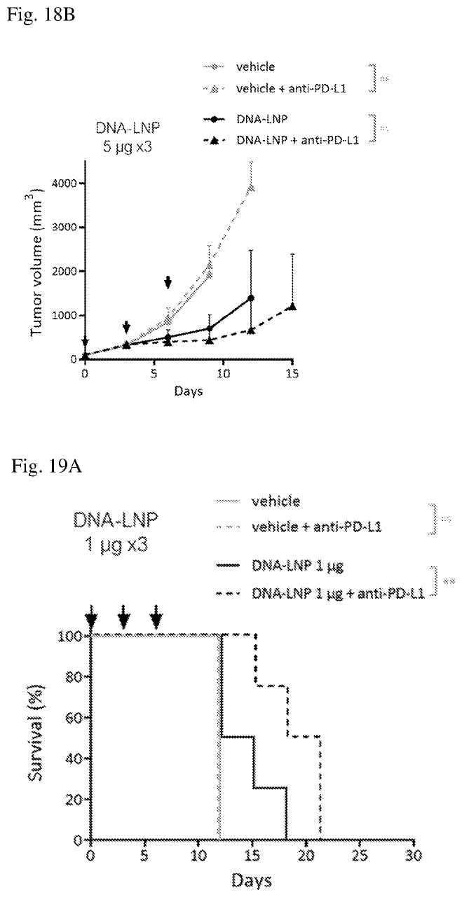

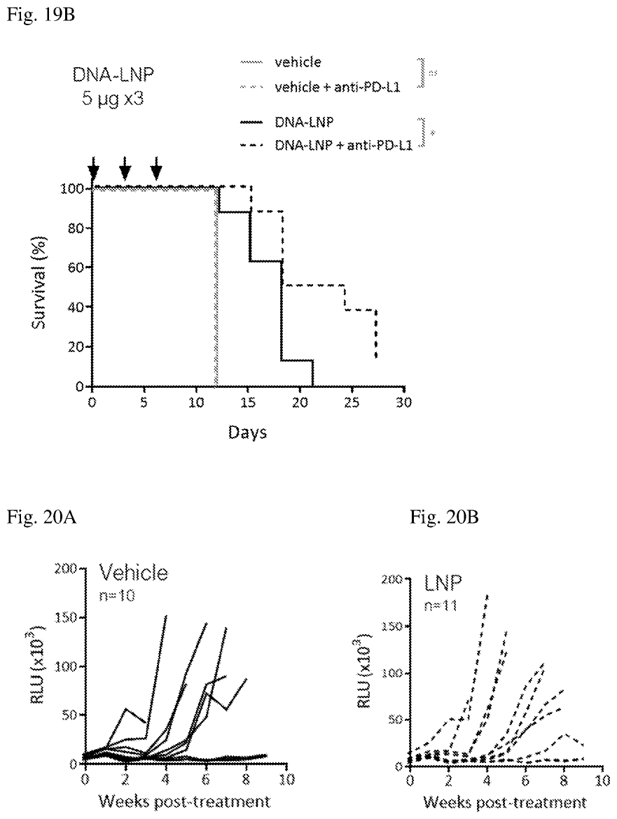

The results illustrate the ability of dsDNA (provided in an LNP) to render a melanoma resistant tumor sensitive to a checkpoint inhibitor (exemplified by anti PD-L1 antibody), providing for a synergistic antitumoral effect. FIGS. 18A and 18B illustrate the ineffectiveness of the checkpoint inhibitor to inhibit tumor growth, the ability of the DNA-LNP to inhibit tumor growth, and the synergistic effect from the combination of DNA-LNP and anti PD-L1 antibody. FIG. 19A and FIG. 19B illustrate the ineffectiveness of the checkpoint inhibitor to increase survival, the ability of the DNA-LNP to increase survival, and the synergistic effect from the combination of DNA-LNP and anti PD-L1 antibody.

### Example 13: Antitumor Effects of Low Dose DNA-LNP in a Liver Cancer Model

A liver cancer model of HCC was established as described in Example 3. Three weeks after HCC induction, administration (vehicle, LNP (equivalent dose to 1 μg DNA-LNP), DNA-LNP (0.2 μg), DNA-LNP (1 μg)) was provided by i.v. administration given one time at week 0. One group was treated with DNA-LNP (0.2 μg) three times, one week apart (week 0, 1, 2). Animal body weight was measured one day after dosing. Serum was collected weekly and assayed for luciferase activity.

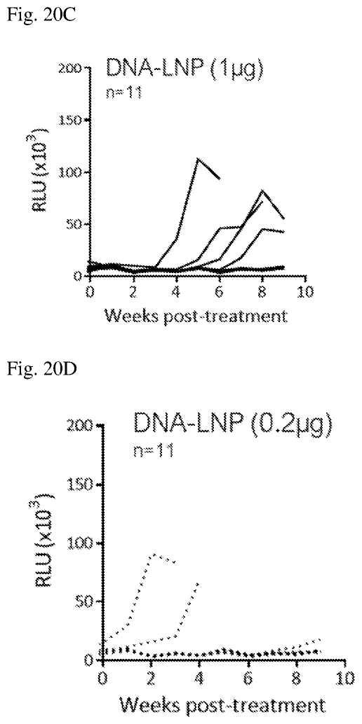

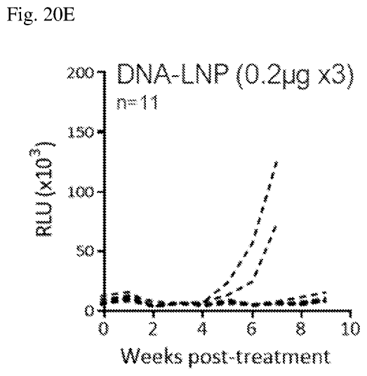

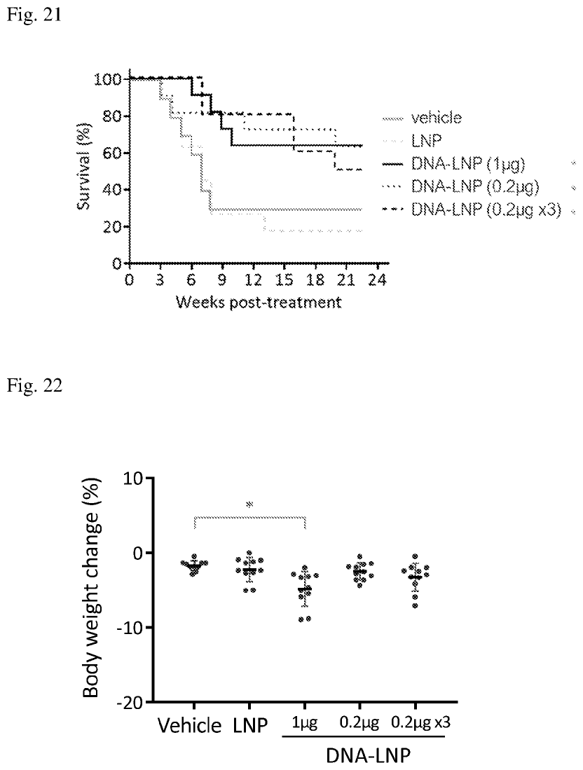

The results illustrate the ability of DNA-LNP to induce an antitumoral effect in a mouse model of HCC without major body weight loss. FIG. 20 illustrates the reduced tumor growth after DNA-LNP administration shown by the reduced serum luciferase activity. FIG. 21 illustrates the ability of DNA-LNP, but not “empty” LNP (no DNA), to increase survival. FIG. 22 illustrates that 0.2 μg of DNA-LNP does not induce body weight loss one day after dosing.

A number of different aspect and embodiments of the instant invention have been described throughout the application. Nevertheless, the skilled artisan without departing from the spirit and scope of the instant invention, can make various changes and modifications of the instant invention to adapt it to various usages and conditions.

1. A method of treating a cancer in a subject comprising administering to said subject:
a) a nanoparticle comprising a double-stranded DNA (dsDNA), wherein said dsDNA comprises a double-stranded region of at least 45 base pairs in length; and
b) a cancer vaccine or a cancer therapeutic agent:
wherein said dsDNA is non-coding or lacks a promoter operatively linked to a region coding for expression in said subject and said nanoparticle is a lipid nanoparticle.

2. The method of claim 1, wherein said cancer is selected from the group consisting of: bladder cancer, breast cancer, colon and rectal cancer, endometrial cancer, kidney cancer, leukemia, liver cancer, lung cancer, melanoma, non-Hodgkin lymphoma, pancreatic cancer, prostate cancer and thyroid cancer.

3. The method of claim 1, wherein said cancer is a tumor.

4. A method of treating leukemia or melanoma in a subject comprising administering to said subject a nanoparticle comprising double-stranded DNA (dsDNA), wherein said dsDNA is at least 45 base pairs in length, provided that if said method treats melanoma, said method further comprises a checkpoint inhibitor.

5. The method of claim 4, wherein said method treats leukemia and said method further comprising the use of a cancer vaccine or cancer therapeutic agent.

6. The method of claim 5, wherein said method comprises administering said vaccine.

7. The method of claim 5, wherein said method comprises administering said therapeutic agent.

8. The method of claim 4, wherein said method treats leukemia or melanoma and further comprises the use of a checkpoint inhibitor.

9. The method of claim 8, wherein said checkpoint inhibitor is selected from the group consisting of: atezolizumab, avelumab, cemiplimab, dostarlimab, durvalumab, nivolumab, ipilimumab, pembrolizumab, an anti-PD-L1 antibody and an anti-PD-1 antibody.

10-11. (canceled)

12. The method of claim 8, wherein said subject is a human.

13. The method of claim 12, wherein said method treats melanoma and said melanoma is resistant to a checkpoint inhibitor treatment in said subject.

14. The method of claim 13, wherein said melanoma is resistant to a PD-L1 inhibitor or a PD-1 inhibitor.

15. The method of claim 14, wherein said checkpoint inhibitor being administered is a PD-L1 inhibitor or a PD-1 inhibitor.

16. (canceled)

17. The method of claim 4, wherein said subject is treated for melanoma and has previously undergone a prior treatment with a checkpoint inhibitor in the absence of a dsDNA-LNP.

18. The method of claim 17, wherein said subject either had no response to said prior treatment or a decreasing level of response to said prior treatment.

19. The method of claim 17, wherein said prior treatment checkpoint inhibitor was a PD-L1 inhibitor or a PD-1 inhibitor.

20. The method of claim 19, wherein said checkpoint inhibitor being administered is a PD-L1 inhibitor or a PD1 inhibitor.

21. (canceled)

22. A method of treating a cancer in a subject comprising administering to said subject:
a) a nanoparticle comprising a double-stranded DNA (dsDNA), wherein said dsDNA comprises a double-stranded region of at least 45 base pairs in length; and
b) a checkpoint inhibitor;
wherein (i) said cancer is resistant to checkpoint inhibitor treatment, (ii) said subject has previously undergone a treatment with a checkpoint inhibitor in the absence of a dsDNA-nanoparticle treatment (iii) said subject either had no response to a prior checkpoint inhibitor treatment in the absence of a dsDNA-nanoparticle or a decreasing level of response to the prior treatment; and/or (iv) said cancer is stratified 13 or 14 based on quantification of CD3+ and CD8+ lymphocyte populations.

23-35. (canceled)

36. The method of claim 1, wherein said vaccine and/or therapeutic agent are administered at, or about, the same time as said dsDNA.

37. The method of claim 4, wherein said dsDNA region is at least 50 base pairs in length.

38. The method of claim 37, wherein said dsDNA region is at least 200 base pairs in length.

39. The method of claim 38, wherein said dsDNA is unmodified.

40-41. (canceled)

42. The method of claim 39, wherein said dsDNA region is provided by two regions of a polynucleotide and said polynucleotide comprises a loop region.

43. The method of claim 39, wherein said dsDNA is non-coding, or lacks a promoter operatively linked to coding region for expression in said subject.

44. The method of claim 4, wherein said nanoparticle is a lipid nanoparticle or lipid polymer nanoparticle.

45. The method of claim 44, wherein said nanoparticle comprises mol % the following components: one or more cationic lipids from about 20% to 65%, one or more phospholipids from about 1% to about 50%, one or more PEG-conjugated lipids from about 0.1% to 10%, and cholesterol from about 0% to about 70%.

46. The method of claim 44, wherein said nanoparticle comprises mol % the following components; one or more cationic lipids from about 20% to 50%, one or more phospholipids from about 5% to about 20%, one or more PEG-conjugated lipids from about 0.1% to 5%, and cholesterol from about 20% to about 60%.

47. (canceled)

48. The method of claim 45, wherein the nanoparticle in mole %, comprises the following components (1) cKK-E12, about 35%; C14-PEG2000, about 2.5%; cholesterol, about 46.5%; and 1,2-dioleoyl-sn-glycero-3-phosphatidyl-ethanolamine (DOPE), about 16%; or (2) Lipid 9, about 50%; C14-PEG2000, about 1.5%; cholesterol, about 38.5%; and 1,2-distearoyl-sn-glycero-3-phosphocholine (DSPC), about 10%.

49. The method claim 1, wherein said nanoparticle comprises mol % the following components; one or more cationic lipids from about 20% to about 50%, one or more phospholipids from about 5% to about 20%, one or more PEG-conjugated lipid from about 0.1% to about 5%, and cholesterol from about 20% to about 60%.

50. The method claim 1, wherein said nanoparticle comprises mol % the following components: one or more cationic lipids from about 20% to 65%, one or more phospholipids from about 1% to about 50%, one or more PEG-conjugated lipid from about 0.1% to about 10%, and cholesterol from about 0% to about 70%.

51. (canceled)

52. The method of claim 1, wherein the nanoparticle in mole %, comprises the following components (1) cKK-E12, about 35%; C14-PEG2000, about 2.5%; cholesterol, about 46.5%; and 1,2-dioleoyl-sn-glycero-3-phosphatidyl-ethanolamine (DOPE), about 16%; or (2) Lipid 9, about 50%; C14-PEG2000, about 1.5%; cholesterol, about 38.5%; and 1,2-distearoyl-sn-glycero-3-phosphocholine (DSPC), about 10%.

53-55. (canceled)

56. A lipid nanoparticle comprising a double-stranded DNA (dsDNA), wherein said dsDNA comprises a double-stranded region of at least 45 base pairs in length and said dsDNA is non-coding or lacks a promoter operatively linked to a region coding for expression in said subject.

57. The lipid nanoparticle of claim 56, wherein said lipid nanoparticle comprises mol % one or more cationic lipids from about 20% to about 65%, one or more phospholipids from about 1% to about 50%, one or more PEG-conjugated lipids from about 0.1% to about 10%, and cholesterol from about 0% to about 70%.

58-59. (canceled)
